(19) United States
(12) Patent Application Publication
(10) Pub. No.: US 2025/0297035 A1
(43) Pub. Date: Sep. 25, 2025

(54) A PEPTIDE AND THE SELECTION METHOD THEREOF

(71) Applicants: THE HONG KONG UNIVERSITY OF SCIENCE AND TECHNOLOGY, Hong Kong (CN); PLERYON THERAPEUTICS (SHENZHEN) LIMITED, Longhua Shenzhen, Guangdong (CN)

(72) Inventors: Ying CHAU, Clearwater Bay, KLN Hong Kong (CN); Rong NI, Clearwater Bay, KLN Hong Kong (CN); Ruilu FENG, Clearwater Bay, KLN, Hong Kong (CN); Yu YU, Yantian, Shenzhen, Guangdong (CN); Zhexun SUN, Yantian, Shenzhen, Guangdong (CN)

(73) Assignees: THE HONG KONG UNIVERSITY OF SCIENCE AND TECHNOLOGY, Hong Kong (CN); PLERYON THERAPEUTICS (SHENZHEN) LIMITED, Longhua Shenzhen, Guangdong (CN)

(21) Appl. No.: 18/709,390

(22) PCT Filed: Nov. 11, 2022
(86) PCT No.: PCT/CN2022/131487

§ 371 Date: May 10, 2024

(30) Foreign Application Priority Data
Nov. 12, 2021 (WO) PCT/CN2021/130257
Dec. 17, 2021 (WO) PCT/CN2021/138963

(51) Int. Cl. C07K 19/00; C12N 15/10

(52) U.S. Cl. CPC C07K 19/00; C12N 15/1093; C07K 2319/735; C07K 2319/80

(57) ABSTRACT

Provided is a peptide, wherein the peptide comprises a first functional module, a second functional module and a third functional module. Provided is a method of selecting a candidate peptide. Provided is a selecting method thereof. The peptide helps deliver nucleic acids efficiently.

### CROSS-REFERENCE TO RELATED APPLICATIONS

This application is a National Stage Application and claims priority under 35 U.S.C. § 371 to Patent Cooperation Treaty application PCT/CN2022/131487, filed Nov. 11, 2022, which claims the benefit of Patent Cooperation Treaty application PCT/CN2021/130257, filed Nov. 12, 2021 and Patent Cooperation Treaty application PCT/CN2021/138963, filed Dec. 17, 2021. Priority is claimed to these applications and the disclosures of these prior applications are considered part of the disclosure of this application and to the extent allowed the entire contents of the aforementioned applications are incorporated herein.

### INCORPORATION BY REFERENCE OF A SEQUENCE LISTING

The present application contains a Sequence Listing which has been submitted electronically as an XML document in the ST.26 format and is hereby incorporated by reference in its entirety. Said Sequence Listing was created on Jan. 2, 2025, is named “5662-105 Sequence Listing.xml,” and is 49,152 bytes in size.

### BACKGROUND OF THE INVENTION

A safe and efficient nucleic acid delivery vehicle has demonstrated great potential in both biological and medical fields. Among these vehicles, lipid-based nanoparticles (LNP) and lipoplexes, dendrimer-based dendriplex and polymer-based polyplexes have been extensively developed as transgene expression agents, but their potential immunogenicity or cytotoxicity, as well as the lack of sequence variation and engineering space, limit their wider application. A new type of nucleic acid delivery vehicle is in need. Viruses are natural organisms that are efficient in delivering genetic materials to cells. For natural viruses, protein capsids are one of the most important structural and functional components that are responsible a wide variety of biological activities related to transfections, including cell entry, endosome escape, nuclear delivery, gene expression etc. To mimic the structure and function of viruses, peptide-based vehicles, or vehicles that composed mainly of peptides, are potential alternatives of the aforementioned non-viral agents.

Peptide is the basic component of viral protein capsids, and thus it is possible to design a large peptide library and screen out suitable delivery vehicles for a specific nucleic acid application, or to design a specific peptide sequence for this nucleic acid delivery application accordingly. However, a functional peptide may compose of a sequence of up to 35 or more amino acids. Since there are more than 500 kinds of natural amino acids, it is technically impossible to screen for suitable peptide sequence by single amino acid variation; while enormous number of unnatural amino acids or non-amino acid components further complicate this picture. Hence, an effective way to construct a manageable-sized peptide (or peptides/non-peptide combo) library and to screen out an effective peptide (or peptide/non-peptide combo) for nucleic acids delivery is in need.

### SUMMARY OF THE INVENTION

The present application provides a peptide, wherein the peptide comprises a first functional module, a second functional module and a third functional module. The present application provides a method of selecting a candidate peptide.

In one aspect, the present application provides a peptide, which comprises a first functional module, a second functional module and a third functional module, wherein the first functional module is able to bind to a nucleic acid, the second functional module is able to self-assemble outside the cell and disassemble inside the cell, and the third functional module is able to be protonated in endosome, wherein the peptide is able to form an assembly with nucleic acid.

In some embodiments, the peptide is able to form a nano-sized assembly with a nucleic acid, the nano-sized assembly is able to enter into a cell, and the delivered exogenous nucleic acid is able to express inside the cell.

In some embodiments, the first functional module is positively charged.

In some embodiments, the first functional module comprises a polypeptide comprising an amino acid comprising a basic amino acid side chain.

In some embodiments, the basic amino acid side chain comprises one or more primary, secondary, tertiary and/or quaternary amine.

In some embodiments, first functional module is able to bind a DNA and/or an RNA.

In some embodiments, the first functional module comprises a natural amino acid and/or an unnatural amino acid.

In some embodiments, the first functional module comprises a polypeptide.

In some embodiments, the first functional module comprises a positively charged amino acid.

In some embodiments, the first functional module comprises one or more lysine and/or arginine.

In some embodiments, the first functional module comprises a nuclear localization peptide.

In some embodiments, the first functional module comprises K, KKK, or a sequence as set forth in any one of SEQ ID NO.3-7.

In some embodiments, the second functional module displays a self-assembly propensity which is able to be tuned by an intracellular or external stimuli.

In some embodiments, the intracellular stimuli comprise various pHs, various temperatures, various redox potentials and/or functional enzymes.

In some embodiments, the second functional module is neutral, and/or is hydrophobic, and/or is able to drive the formation of beta sheets before encountering the intracellular stimuli.

In some embodiments, the second functional module is charged, and/or is less hydrophobic, and/or is able to disassemble the beta sheets after encountering the intracellular stimuli.

In some embodiments, the self-assembly propensity of the second functional module is able to be tuned by at least one intracellular or external stimuli.

In some embodiments, the self-assembly propensity of the second functional module is able to be tuned by at least two intracellular stimuli.

In some embodiments, the intracellular stimuli comprise a change in redox potential and a change in pH.

In some embodiments, the second functional module comprises a polypeptide.

In some embodiments, the second functional module comprises a natural amino acid and/or an unnatural amino acid.

In some embodiments, the second functional module comprises at least one amino acid that comprises a non-polar side chain.

In some embodiments, the second functional module comprises at least one amino acid that comprises a side chain which contains a disulfide bond.

In some embodiments, the amino acid that comprises a side chain which contains a disulfide bond is t-butyl-s-s-cysteine (C_{stBu}).

In some embodiments, the second functional module comprises at least one amino acid that comprises an imidazole side chain.

In some embodiments, the amino acid that comprises an imidazole side chain is a histidine.

In some embodiments, the second functional module comprises one or more alanine, asparagine, cysteine, glutamine, histidine, isoleucine, leucine, methionine, phenylalanine, serine, threonine, tryptophan, tyrosine, valine, S-Benzyl-L-cysteine (C_{bzyl}), t-butyl-s-s-cysteine (C_{stBu}) and/or the combination thereof.

In some embodiments, the second functional module comprises a sequence as set forth in any one of SEQ ID NO.8-11.

In some embodiments, the third functional module is protonated at pH lower than 7.4.

In some embodiments, the third functional module comprises a polypeptide.

In some embodiments, the third functional module comprises a natural amino acid and/or an unnatural amino acid.

In some embodiments, the third functional module comprises at least one amino acid comprising an imidazole side chain.

In some embodiments, the third functional module comprises one or more copies of an amino acid that comprises an imidazole side chain (for example, Histidine (H)).

In some embodiments, the third functional module comprises H, HH, or a sequence as set forth in SEQ ID NO.14.

In some embodiments, the peptide comprises a fourth functional module, and the fourth functional module comprises a flexible linker.

In some embodiments, the fourth functional module is hydrophobic or hydrophilic.

In some embodiments, the fourth functional module comprises a polypeptide and/or a non-peptide.

In some embodiments, the fourth functional module comprises a natural amino acid and/or an unnatural amino acid.

In some embodiments, the fourth functional module comprises a carbon chain of 2-20 carbons or a polyethylene glycol.

In some embodiments, the fourth functional module comprises 3-aminopropanoic acid (C₃), 6-aminohexanoic acid (C₆), 12-aminododecanoic acid (C₁₂), or 16-aminohexadecanoic acid (C₁₆).

In some embodiments, the peptide comprises a fifth functional module, and the fifth functional module comprises a hydrophobic end moiety.

In some embodiments, the peptide wherein at least one amino acid at the end of the fifth functional module comprises an aromatic group.

In some embodiments, the aromatic group comprises a Fmoc group.

In some embodiments, the peptide comprises a sixth functional module, and the sixth functional module is hydrophilic.

In some embodiments, the sixth functional module comprises a polar and/or negatively charged group.

In some embodiments, the sixth functional module comprises a polypeptide or a non-peptide.

In some embodiments, the sixth functional module comprises one or more serine, tyrosine, threonine, asparagine, glutamine, aspartic acid, glutamic acid and/or the combination thereof.

In some embodiments, the sixth functional module comprises a hydrophilic polymer.

In some embodiments, the hydrophilic polymer comprises a polyethylene glycol and/or a polysaccharide.

In some embodiments, the sixth functional module comprises GSP, D, or a sequence as set forth in SEQ ID NO.21.

In some embodiments, the fifth functional module locates at a terminal of the peptide.

In some embodiments, the sixth functional module locates at a terminal of the peptide.

In some embodiments, the order of the first functional module to the fourth functional module is arbitrary.

In some embodiments, the peptide comprises one or more functional module selected from the first functional module to the sixth functional module.

In some embodiments, the peptide comprises PKKKRKVG-HHHH(SEQ ID NO:23)-C₁₂-LLHC_{Bzyl}C_{Bzyl}HLL GSPHHD(SEQ ID NO:34), RKKRRQRRR-HHHH(SEQ ID NO:24)-C₁₂-LLHC_{Bzyl}C_{Bzyl}HLL GSPHHD (SEQ ID NO:35), Fmoc-PKKKRKVG(SEQ ID NO:25)-C₁₂-HHHH-C_{stBu}HHC_{stBu}C_{stBu}HHC_{stBu}GSPHHD(SEQ ID NO:36), or Fmoc-C₁₂-PKKKKKVG-HHHH-C_{stBu}HHC stBuC_{stBu}HHC_{stBu}GSPHHD (SEQ ID NO 26).

In other aspect, the present application provides a method of selecting a candidate peptide, wherein the method comprises: Preparing a library of the candidate peptide, wherein the candidate peptide comprises at least two kinds of functional module, and each the functional module is respectively selected from a corresponding library of functional module wherein the corresponding library of functional module comprises at least two different functional module sequences.

In some embodiments, the library of functional module comprises a first functional module library, and the first functional module library comprises at least two different first functional module sequences.

In some embodiments, the first functional module library comprises at least 10³ first functional module sequences.

In some embodiments, the first functional module library is obtained by chemical synthesis.

In some embodiments, the first functional module is able to bind the nucleic acid.

In some embodiments, the first functional module is able to bind a DNA and/or an RNA.

In some embodiments, the first functional module is positively charged.

In some embodiments, the first functional module comprises polypeptides comprising a basic amino acid side chain.

In some embodiments, the basic amino acid side chain comprises one or more primary, secondary, tertiary and/or quaternary amine.

In some embodiments, the first functional module comprises a natural amino acid and/or an unnatural amino acid.

In some embodiments, the first functional module comprises a positively charged amino acid.

In some embodiments, the first functional module comprises one or more lysine and/or arginine.

In some embodiments, the first functional module comprises a nuclear localization peptide.

In some embodiments, in the first functional module library, at least 50% first functional module sequences comprise at least two consecutive lysine.

In some embodiments, in the first functional module library, at least 30% first functional module sequences comprise at least three consecutive lysine.

In some embodiments, the first functional module comprises K, KKK, or a sequence as set forth in any one of SEQ ID NO.3-7.

In some embodiments, the library of functional module comprises a second functional module library, and the second functional module library comprises at least two different second functional module sequences.

In some embodiments, the second functional module library comprises at least 10³ second functional module sequences.

In some embodiments, the second functional module library is obtained by chemical synthesis.

In some embodiments, in the second functional module library, at least 50% of second functional module sequences comprise at least two amino acids that comprise a side chain which contains a disulfide bond and/or at least two amino acids that comprise an imidazole side chain.

In some embodiments, the second functional module is able to self-assemble outside the cell and disassemble inside the cell.

In some embodiments, the second functional module comprises a self-assembly propensity which is able to be tuned by an intracellular or external stimuli.

In some embodiments, the intracellular stimuli comprise various pHs, various temperatures, various redox potentials and/or functional enzymes.

In some embodiments, the second functional module is neutral, and/or is hydrophobic, and/or is able to drive the formation of beta sheets before encountering the intracellular stimuli.

In some embodiments, the second functional module carries a charge, and/or is less hydrophobic, and/or is able to disassemble the beta sheets after encountering the intracellular stimuli.

In some embodiments, the self-assembly propensity of the second functional module is able to be tuned by at least one intracellular or external stimuli.

In some embodiments, the self-assembly propensity of the second functional module is able to be tuned by at least two intracellular stimuli.

In some embodiments, the intracellular stimuli comprise a change in redox potential and/or a change in pH.

In some embodiments, the second functional module comprises a polypeptide.

In some embodiments, the second functional module comprises a natural amino acid and/or an unnatural amino acid.

In some embodiments, the second functional module comprises at least one amino acid that comprises a non-polar side chain.

In some embodiments, the second functional module comprises at least one amino acid that comprises a side chain which contains a disulfide bond.

In some embodiments, the amino acid that comprises a side chain which contains a disulfide bond is t-butyl-s-s-cysteine (C_{stBu}).

In some embodiments, the second functional module comprises at least one amino acid that comprises an imidazole side chain.

In some embodiments, the amino acid that comprises an imidazole side chain is a histidine.

In some embodiments, the second functional module comprises one or more alanine, asparagine, cysteine, glutamine, histidine, isoleucine, leucine, methionine, phenylalanine, serine, threonine, tryptophan, tyrosine, valine, S-Benzyl-L-cysteine (C_{bzyl}), t-butyl-s-s-cysteine (C_{stBu}) and/or the combination thereof.

In some embodiments, the second functional module comprises a sequence as set forth in any one of SEQ ID NO.8-11.

In some embodiments, the library of functional module comprises a third functional module library, and the third functional module library comprises at least two different third functional module sequences.

In some embodiments, the third functional module library comprises at least 10³ third functional module sequences.

In some embodiments, the third functional module library is obtained by chemical synthesis.

In some embodiments, in the third functional module library, at least 50% third functional module sequences sequences comprise at least two consecutive amino acids that comprise a side chain which contains an imidazole.

In some embodiments, in the third functional module library, at least 30% third functional module sequences sequences comprise at least four consecutive amino acids that comprise a side chain which contains an imidazole.

In some embodiments, the third functional module is able to be protonated inside endosome.

In some embodiments, the third functional module is protonated at pH lower than 7.4.

In some embodiments, the third functional module comprises a polypeptide.

In some embodiments, the third functional module comprises a natural amino acid and/or an unnatural amino acid.

In some embodiments, the third functional module comprises one or more copies of an amino acid that comprises an imidazole side chain (for example, Histidine (H)).

In some embodiments, the third functional module comprises H, HH, or a sequence as set forth in SEQ ID NO.14.

In some embodiments, the library of functional module comprises a fourth functional module library, and the fourth functional module library comprises at least two different fourth functional module sequences.

In some embodiments, the fourth functional module library comprises at least 10² fourth functional module sequences.

In some embodiments, the fourth functional module library is obtained by chemical synthesis.

In some embodiments, in the fourth functional module library, at least 50% of the fourth functional module sequence comprises a carbon chain comprising at least three consecutive carbons.

In some embodiments, in the fourth functional module library, at least 30% of the fourth functional module sequence comprises a carbon chain comprising at least ten consecutive carbons.

In some embodiments, the fourth functional module comprises a linker.

In some embodiments, the fourth functional module is hydrophobic or hydrophilic.

In some embodiments, the fourth functional module comprises a polypeptide and/or a non-peptide.

In some embodiments, the fourth functional module comprises a natural amino acid and/or an unnatural amino acid.

In some embodiments, the fourth functional module comprises a carbon chain of 2-20 carbons or a polyethylene glycol.

In some embodiments, the fourth functional module comprises 3-aminopropanoic acid (C₃), 6-aminohexanoic acid (C₆), 12-aminododecanoic acid (C₁₂), or 16-aminohexadecanoic acid (C₁₆).

In some embodiments, the library of functional module comprises a fifth functional module library, and the fifth functional module library comprises at least two different fifth functional module sequences.

In some embodiments, the fifth functional module library comprises at least 10² fifth functional module sequences.

In some embodiments, the fifth functional module library is synthesized.

In some embodiments, the fifth functional module comprises a hydrophobic end moiety.

In some embodiments, at least one amino acid at the end of the fifth functional module sequence comprises an aromatic group.

In some embodiments, the aromatic group comprises a Fmoc group.

In some embodiments, the library of functional module comprises a sixth functional module library, and the sixth functional module library comprises at least two different sixth functional module sequences.

In some embodiments, the sixth functional module library comprises at least 10² sixth functional module sequences.

In some embodiments, the sixth functional module library is obtained by chemical synthesis.

In some embodiments, in the sixth functional module library, at least 90% of the sixth functional module sequences do not form any secondary structure.

In some embodiments, in the sixth functional module library, at least 99% of the sixth functional module sequences are hydrophilic.

In some embodiments, the sixth functional module is hydrophilic.

In some embodiments, the sixth functional module comprises a polar and/or negatively charged group.

In some embodiments, the sixth functional module comprises a polypeptide or a non-peptide.

In some embodiments, the sixth functional module comprises one or more serine, tyrosine, threonine, asparagine, glutamine, aspartic acid, glutamic acid and/or the combination thereof.

In some embodiments, the sixth functional module comprises a hydrophilic polymer.

In some embodiments, the hydrophilic polymer comprises a polyethylene glycol and/or a polysaccharide.

In some embodiments, the sixth functional module comprises GSP, D, or a sequence as set forth in SEQ ID NO.21.

In some embodiments, the library of the candidate peptide comprises at least 10⁵ candidate peptide sequences.

In some embodiments, the library of the candidate peptide is obtained by chemical synthesis.

In some embodiments, the candidate peptide comprises at least one copy of the first functional module sequence.

In some embodiments, the candidate peptide comprises at least one copy of the second functional module sequence.

In some embodiments, the candidate peptide comprises at least one copy of the third functional module sequence.

In some embodiments, the candidate peptide comprises at least one copy of the fourth functional module sequence.

In some embodiments, the candidate peptide comprises at least one copy of the fifth functional module sequence.

In some embodiments, the candidate peptide comprises at least one copy of the sixth functional module sequence.

In some embodiments, in the candidate peptide library, the order of the first functional module to the fourth functional module is arbitrary.

In some embodiments, the fifth functional module locates at the terminal of the candidate peptide.

In some embodiments, the sixth functional module locates at the terminal of the candidate peptide.

In other aspect, the present application provides the peptide, which is prepared by the method.

In other aspect, the present application provides a use of the peptide in preparing a nucleic acid-peptide co-assembly.

In some embodiments, the nucleic acid comprises a DNA, and/or an RNA.

In some embodiments, the RNA comprises a mRNA.

Additional aspects and advantages of the present application will become readily apparent to those skilled in this art from the following detailed description, wherein only illustrative embodiments of the present application are shown and described. As will be realized, the present application is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates the fluorescence microscope images of transfected cells for peptide_1, peptide_3, peptide_5 and peptide_12.

FIG. 2 illustrates the fluorescence microscope images of transfected cells for peptide_12 and peptide_13.

FIGS. 3A-3D illustrate the results of optimization of component (peptide and DNA) concentration in DMEM

FIGS. 4A-4B illustrate the cell internalization efficiency of DNA by peptide-DNA co-assemblies with varied DNA concentration and varied peptide concentration.

FIGS. 5A-5B illustrate the cell internalization efficiency by peptide-DNA co-assemblies with 24 hr- and 48 hr-incubation.

FIG. 6 illustrates the result of gel electrophoresis of peptide-DNA co-assemblies.

FIGS. 7A-7B illustrate the transfection efficiency with the peptide-mRNA co-assemblies and quantification of the transfected cells.

FIG. 8 illustrates transfection efficiency of the peptide-mRNA co-assemblies in different cell lines.

FIG. 9 illustrates the fluorescence images of Hela cells were transfected with the peptide_12-mRNA co-assemblies and peptide_13-mRNA co-assemblies.

FIGS. 10A-10B illustrate the TEM image and DLS results of peptide_13-mRNA co-assemblies at N/P ratio of 0.8, 2, 4, 8 or 16.

FIG. 11A illustrates the gel electrophoresis of peptide_13-mRNA co-assemblies under different N/P ratios was estimated with agarose gel electrophoresis assay.

FIG. 11B illustrates the gel electrophoresis of mRNA by peptide_13-mRNA against serum and enzymatic degradation at N/P=2.

FIG. 12 illustrates the cytotoxicity effect of the peptide_13 on HeLa, MCF-7, SK-N-MC, HEK293 and RAW264.7 cells.

FIGS. 13A-13B illustrate the cytocompatibility and transfection efficiency of peptide_13-mRNA co-assemblies in HeLa cells.

FIG. 14 illustrates the endosomal escape of mRNA. The intracellular trafficking of the peptide_13-mRNA co-assembly was observed by live-cell confocal microscope (FIG. 14A) and quantified both the line profiles (FIG. 14B) and the Mander's overlap coefficient M130 (FIG. 14C).

FIGS. 15A-15B illustrate the transfection efficiencies of the co-assemblies under optimal conditions in various cell lines.

FIGS. 16A-16B illustrate the stability of peptide_13-mRNA co-assemblies after lyophilization.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

In the present application, the term “peptide” generally refers to any of a group of compounds comprising two or more amino acids linked by chemical bonding between their respective carboxyl and amino groups. The peptide may comprise peptides and proteins that are of sufficient length and composition to affect a biological response, for example, assemble and/or deliver a DNA, and/or an RNA. The peptide may comprise a modified amino acid. The peptide may comprise a polypeptide, which comprises a group of natural or synthetic polymers made up of amino acids chemically linked together such as peptides linked together. The peptide of the present application may comprise at least one functional module, which means that every functional module may have a respective biological and/or chemical function itself, and may endow this function to the peptide.

In the present application, the term “first functional module” generally refers to a functional module of a peptide which may be able to bind to a nucleic acid. The first functional module may comprise a basic amino acid side chain. For example, the basic amino acid side chain may carry positive charge at neutral pH. For example, the amino acid may comprise arginine (Arg) and lysine (Lys). In the present application, the first functional module may be able to bind to nucleic acids, which may be negatively charged. For example, the first functional module may be positively charged. In the present application, the first functional module may be able to localize nucleus. The first functional module may comprise a nuclear localization signal (NLS) sequence, which may be responsible for translocating a nucleic acid and/or a protein into the nucleus. The first functional module may comprise a nuclear localization peptide. The first functional module may be able to bind to a nucleic acid molecule.

In the present application, the term “second functional module” generally refers to a functional module of a peptide which is able to self-assemble. In the present application, the “self-assemble” may be a process of spontaneous assembling into an ordered nanostructure. For example, the self-assemble may be tunable. For example, the self-assemble may mean being able to self-assemble outside the cell and disassemble inside the cell. For example, the self-assembly propensity may be able to be tuned by an intracellular or external stimuli. For example, the second functional module may comprise hydrophobic amino acid side chain. For example, the second functional module may comprise an alternating hydrophilic and hydrophobic amino acid residue. For example, the second functional module may comprise a peptide, and/or a non-peptide compound.

In the present application, the term “third functional module” generally refers to a functional module of a peptide which is able to facilitate nucleic acids to escape from an endosome. In the present application, the endosome may be referred to a membranous organelle to which molecules internalized by a cell via endocytosis are transferred. For example, the endosome may comprise a lysosome. For example, the endosome may comprise lysozyme, including MIIC, CUV, melanosomes, secretory granules, soluble granules, lysosome-related organelle (for example platelet-dense granules, basophilic granules), Birbeck granules, phagolysosomes, and/or secretory lysosomes. For example, the endosome may be able to break down many kinds of biomolecules (for example, a nucleic acids and/or a protein). For example, the third functional module may be protonated at pH lower than 7.4, for example, may be protonated at pH lower than 7, 6.5, 6, 5.5, 5, 4.5 or 4.

In the present application, the term “nuclear localization peptide” generally refers to a peptide which comprises an amino acid sequence that ‘tags’ a sequence for importing cargoes into the cell nucleus by nuclear transportation. The nuclear localization peptide may comprise a nuclear localization signal or sequence (NLS). The nuclear localization peptide may be classified into classical and non-classical. For example, the nuclear localization peptide may comprise a sequence PKKKRKV (SEQ ID NO:40) in a SV40 Large T-antigen.

In the present application, the term “unnatural amino acid” generally refers to “non-naturally encoded amino acid”, “non-natural amino acid”, “non-naturally occurring amino acid”, and their various hyphens. The unnatural amino acid may comprise amino acids that do not occur naturally and are obtained synthetically or by modification of unnatural amino acids. For example, the unnatural amino acid may be an amino acid that is not one of the usual 20 amino acids and is not pyrrolysine or selenocysteine.

In the present application, the term “stimuli” generally refers to a physical or chemical change in the environment that results in a response by a stimulus-responsive functional module of the present application. For example, the stimuli may comprise temperature changes, conductivity changes, and/or pH changes.

In the present application, the term “fourth functional module” generally refers to a functional module of a peptide which is able to link at least two functional modules of the present application. For example, the fourth functional module may comprise a linker. For example, the fourth functional module may link at least two functional modules which require a certain degree of movement or interaction. For example, the fourth functional module may comprise a peptide and/or a compound.

In the present application, the term “hydrophobic” generally refers to the property of lacking affinity for, or even repelling, water. For example, the more hydrophobic an agent (for example, a functional module), the more that agent tends to not dissolve in, not mix with, or not be wetted by water. Hydrophilicity and hydrophobicity can be spoken of in relative terms, such as but not limited to a spectrum of hydrophilicity/hydrophobicity within a group of compounds.

In the present application, the term “hydrophilic” generally refers to the property of having affinity for water. For example, the hydrophilic may relate to a moiety (for example, a hydrophobic moiety) of an agent (for example, a functional module), which may comprise an ionizable, polar, or polarizable atom, or which otherwise may be solvated by water molecules.

In the present application, the term “hydrophobic moiety” generally refers to a moiety, which has the property of lacking affinity for water. For example, the hydrophobic moiety may be an aliphatic hydrocarbon chain and/or a cyclic compound that has no positive or negative charge and can be bound to the molecule by hydrophobic interactions; For example, the hydrophobic moiety may comprise alkyl, benzyl, phenyl, propyl, butyl, indole, S-methyl thioether, methyl, alkenyl, alkynyl, and aryl moieties. For example, the hydrophobic moiety may be unsubstituted or substituted where chemically possible (for example, not hydrogen). In one embodiment, the hydrophobic moiety may be substituted or unsubstituted phenyl. Examples of substituents may comprise alkyl, alkenyl, alkynyl, alkoxy, halogen, amino, thiol, hydroxy, nitro, aryl, and heteroaryl.

In the present application, the term “aromatic group” generally refers to an aromatic compound which may have a conjugated cyclic hydrocarbon that conforms to the Huckel (4n+2) rule when n is an integer from 1 to about 5. For example, the aromatic group may be monocyclic and polycyclic. For example, the aromatic group may be found in Morrison and Boyd, Organic Chemistry, (5th Ed., 1987), Chapter 13, entitled “Aromaticity,” pages 477-497, incorporated herein by reference.

In the present application, the term “Fmoc group” generally refers to a fluorenyl methoxycarbonyl protecting group. The Fmoc group may be a base-labile protecting group used in organic synthesis. The Fmoc group protected amino acid may have the following structure:

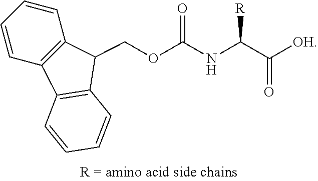

In the present application, the term “fifth functional module” generally refers to a functional module of a peptide which is able to increase hydrophobicity of the peptide of the present application. For example, the fifth functional module may be at the end of the peptide and link to the first functional module. For example, the fifth functional module may be at the N terminal or C terminal of the peptide of the present application. For example, the fifth functional module may comprise a chemical group.

In the present application, the term “sixth functional module” generally refers to a functional module of a peptide which is able to increase hydrophilicity of the peptide of the present application. For example, the sixth functional module may be at the end of the peptide and link to the second functional module. For example, the sixth functional module may be at the N terminal or C terminal of the peptide of the present application. For example, the sixth functional module may comprise a peptide, a compound (for example, a hydrophilic polymer) and/or a chemical group.

In the present application, the term “candidate peptide” generally refers to a peptide which may be regarded as a functional peptide (for example, through a test) with a desired biological function (for example, delivering a nucleic acid into a cell). In the present application, the candidate peptide may exist in a library of the candidate peptide. For example, the library of the candidate peptide may comprise at least 103 different candidate peptides. The candidate peptide may comprise at least one functional module of the present application.

In the present application, the term “a corresponding library of functional module” generally refers to a library comprising at least two different functional module sequences of one kind of the functional module. For example, a library of the first functional module may comprise at least two different first functional module sequences. The library of the first functional module may not comprise any other functional module other than the first functional module. For example, the library of the first functional module may not comprise any second, third, fourth, fifth and/or sixth functional module of the present application. In the present application, the corresponding library of functional module may be built to provide various (for example, at least two) candidate functional modules of one kind of the functional module.

In the present application, the term “functional module sequence” generally refers to a candidate peptide sequence which belongs to a specific functional module in the corresponding library of functional module. For example, in the first functional module library, there may exist at least two different peptide sequences of the first functional module, which means the functional and/or structure of the first functional module sequences are identical to that of the first functional module.

In the present application, the term “chemical synthesis” generally refers to an artificial execution of useful chemical reactions to obtain one or several products. The chemical synthesis may be occurred by physical and chemical manipulations usually involving one or more reactions. The chemical synthesis may comprise a series of individual chemical reactions.

In the present application, the term “nucleic acid-peptide assembly” generally refers to an assembly in which the peptide is able to bind nucleic acids. For example, the peptide thereof may be able to deliver a large panel of cargos (plasmid DNA, oligonucleotide, siRNA, mRNA, small activation RNA, self-amplifying RNA, cRNA . . . ) into a wide variety of cell types in vitro and/or in vivo.

In the present application, the term “nucleic acid” generally refers to oligonucleotide or polynucleotide such as deoxyribonucleic acids acid (DNA) and/or ribonucleic acids acid (RNA) as well as analogs of either RNA or DNA, any of which are in single or double stranded form.

### Peptide

In one aspect, the present application provides a peptide, which comprises a first functional module, a second functional module and a third functional module, wherein the first functional module is able to bind a nucleic acid, the second functional module is able to self-assemble outside the cell and disassemble inside the cell, and the third functional module is able to be protonated in endosome, wherein the peptide is able to form an assembly with nucleic acid.

For example, the peptide may be able to form a nano-sized assembly with a nucleic acid. For example, the nano-sized assembly may be able to enter into a cell, and the delivered exogenous nucleic acids may be able to express inside the cell.

For example, the first functional module may be positively charged.

For example, the first functional module may comprise polypeptides comprising a basic amino acid side chain. For example, the basic amino acid side chain may comprise arginine (Arg) and/or lysine (Lys).

For example, the basic amino acid side chain may comprise one or more primary, secondary, tertiary and/or quaternary amine.

In the present application, the first functional module may be able to bind a DNA and/or an RNA. For example, the first functional module may bind the nucleic acid (such as a DNA and/or an RNA) by the interaction between positively charged module and negatively charged nucleic acids.

In the present application, the first functional module may comprise one or more lysine and/or arginine. For example, the first functional module may comprise at least two (for example, at least three, at least four, at least five or more) successive lysine and/or arginine.

In the present application, the first functional module may comprise a nuclear localization peptide. In the present application, the nuclear localization peptide may comprise a nuclear localization sequence (NLS) and/or a nuclear localization signal (NLS). For example, the NLS may mediate the transportation of proteins from the cytoplasm into the nucleus. For example, the NLS may comprise 4-8 basic amino acids, for example, may comprise generally 4 or more positively charged residues, that is, arginine (R) or lysine (K). For example, the NLS may comprise seven amino acids, Pro-Lys-Lys-Lys-Arg-Lys-Val (PKKKRKV; (SEQ ID NO:40). For example, the NLS may comprise a sequence shown in Table 1 of Lu et al. Cell Commun Signal (2021) 19:60. For example, the first functional module may comprise a sequence of PKKKRKVG (SEQ ID NO:5).

In the present application, the first functional module may be able to bind nucleic acids. For example, the first functional module may be positively charged. For example, the first functional module may bind a nucleic acid by the interaction between positively charged peptide and negatively charged nucleic acid. For example, the nucleic acid may comprise a DNA and/or an RNA. For example, the nucleic acid may comprise a single chain and/or a double chain.

In the present application, the first functional module may comprise a natural amino acid and/or an unnatural amino acid. For example, the first functional module may comprise a positively charged amino acid.

For example, the natural amino acid may comprise alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and/or valine. For example, the unnatural amino acid may comprise D-amino acids, homo amino acids, N-methyl amino acids, alpha-methyl amino acids, beta (homo) amino acids, gamma amino acids, helix/turn stabilizing motifs, and/or the ones with backbone modifications (e.g. peptoids).

In the present application, the first functional module may comprise K, KKK, or a sequence as set forth in any one of SEQ ID NO.3-7.

In the present application, the second functional module may comprise a self-assembly propensity which is able to be tuned by an intracellular or external stimuli. In the present application, the intracellular stimuli may comprise various pHs, various temperatures, various redox potentials and/or functional enzymes.

In the present application, the self-assembly propensity may be changed with the change of the intracellular or external stimuli. For example, the self-assembly propensity may be reversible. For example, when the pH is higher, the second functional module may be self-assembled and may form a secondary structure (for example, a beta sheet); when the pH is lower, the secondary structure may be disrupted (i.e. a non-beta sheet structure). For example, when the redox potential is lower, the second functional module may be self-assembled and may form a secondary structure (for example, a beta sheet); when the redox potential is higher, the secondary structure may be disrupted (i.e. a non-beta sheet structure).

The self-assembly propensity may be important in aggregation of a peptide. In the present application, the self-assembly propensity may be evaluated by molecular dynamics (MD) simulation.

In the present application, the self-assembly propensity of the second functional module may be able to be tuned by at least one (for example, at least 2, at least 3, at least 4, at least 5 or more) intracellular or external stimuli.

In the present application, the self-assembly propensity of the second functional module may be able to be tuned by at least two (for example, at least 3, at least 4, at least 5 or more) intracellular stimuli. For example, the intracellular stimuli may comprise a change in redox potential and a change in pH.

In the present application, the second functional module may comprise a polypeptide.

In the present application, the second functional module may be neutral, and/or may be hydrophobic, and/or may be able to drive the formation of beta sheets before encountering the intracellular stimuli. For example, the beta sheet may consist of beta strands (β-strands) connected laterally by at least two or three backbone hydrogen bonds, forming a generally twisted, pleated sheet. For example, large aromatic residues (for example, tyrosine, phenylalanine, and/or tryptophan) and/or β-branched amino acids (for example, threonine, valine, and/or isoleucine) may be favored to be found in β-strands in the middle of the beta sheets.

In the present application, the second functional module may be charged, and/or may be less hydrophobic, and/or disassembles the beta sheets after encountering the intracellular stimuli.

In the present application, the second functional module may comprise at least one, at least two, at least three or at least four amino acids that comprise a non-polar side chain.

In the present application, the second functional module may comprise at least one, at least two, at least three or at least four amino acids that comprise a side chain which contains a disulfide bond.

In the present application, the amino acid that comprises a side chain which contains a disulfide bond may be t-butyl-s-s-cysteine (C_{stBu}).

In the present application, the second functional module may comprise at least one, at least two, at least three or at least four amino acids that comprise an imidazole side chain.

In the present application, the amino acid that comprises an imidazole side chain may be a histidine.

In the present application, the second functional module may comprise a natural amino acid and/or an unnatural amino acid. For example, the second functional module may comprise one or more alanine, asparagine, cysteine, glutamine, histidine, isoleucine, leucine, methionine, phenylalanine, serine, threonine, tryptophan, valine, S-Benzyl-L-cysteine (C_{bzyl}), t-butyl-s-s-cysteine (C_{stBu}), ethyl-cysteine disulfide, 1-n-propyl cysteine disulfide, 1-n-butyl cysteine disulfide, 1-n-pentyl cysteine disulfide, phenyl-s-s-cysteine, benzyl-s-s-cysteine and/or the combination thereof.

In the present application, the second functional module may comprise a sequence as set forth in any one of SEQ ID NO.8-11.

In the present application, the third functional module may be protonated at pH lower than 7.4. For example, the third functional module may be protonated at pH lower than 7, lower than 6.5, lower than 6, lower than 5.5, lower than 5, lower than 4.5, lower than 4 or lower.

For example, the third functional module may cause membrane disruption at pH lower than 7, lower than 6.5, lower than 6, lower than 5.5, lower than 5, lower than 4.5, lower than 4 or lower.

In the present application, the third functional module may comprise a natural amino acid and/or an unnatural amino acid.

In the present application, the third functional module may comprise one or more copies of Histidine (H). For example, the pH sensitivity of the third functional module may be determined by histidine substitution numbers.

In the present application, the third functional module may comprise H, HH, or a sequence as set forth in SEQ ID NO.14.

In the present application, the peptide may comprise a fourth functional module, and the fourth functional module may comprise a linker.

In the present application, the fourth functional module may comprise a natural amino acid and/or an unnatural amino acid. For example, the unnatural amino acid may comprise an amino fatty acid.

In the present application, the fourth functional module may comprise a carbon chain of 2-20 carbons (for example, 2, 3, 4, 5, 6, 7, 8, 9, 10, 11, 12, 13, 14, 15, 16, 17, 18, 19 or 20) or a polyethylene glycol. For example, the fourth functional module may be a carbon chain with a modification.

In the present application, the fourth functional module may comprise 3-aminopropanoic acid, (C₃), 6-aminohexanoic acid (C₆), 12-aminododecanoic acid (C₁₂), or 16-aminohexadecanoic acid (C₁₆).

In the present application, the peptide may comprise a fifth functional module, and the fifth functional module may comprise an end group comprising a hydrophobic moiety.

In the present application, the peptide wherein at least one amino acid at the end of the fifth functional module may comprise an aromatic group. For example, the aromatic group may comprise a benzene-based ring, and/or a heteroarene, which follows Hückel's rule (for monocyclic rings: when the number of its π electrons equals 4n+2, where n =0, 1, 2, 3, . . . ).

In the present application, the aromatic group may comprise a Fmoc group. For example, the fifth functional module may help to increase hydrophobicity of the peptide. For example, the fifth functional module may locate at the terminal (for example, the N terminal or the C terminal) of the peptide of the present application.

In the present application, the peptide may comprise a sixth functional module, and the sixth functional module is hydrophilic.

In the present application, the sixth functional module may comprise a polar and/or negatively charged group.

In the present application, the sixth functional module may comprise a polypeptide or a non-peptide.

In the present application, the sixth functional module may comprise one or more serine, tyrosine, threonine, asparagine, glutamine, aspartic acid, glutamic acid and/or the combination thereof.

In the present application, the sixth functional module may comprise a hydrophilic polymer.

In the present application, the hydrophilic polymer may comprise a polyethylene glycol and/or a polysaccharide. For example, the polyethylene glycol may comprise PEG3000, PEG4000, PEG1000, PEG3350, PEG200, PEG8000, PEG600, PEG400, PEG300, PEG1500, PEG2000, O-(2-Carboxyethyl) polyethylene glycol, PEG2000, Polyethylene glycol dimethyl ether 500, and/or O-(2-Aminoethyl) polyethylene glycol. For example, the polysaccharide may comprise a homo-polysaccharide and a hetero-polysaccharide.

For example, the sixth functional module may help to enhance the solubility of the peptide of the present application. For example, the sixth functional module may locate at the terminal (for example, the N terminal or the C terminal) of the peptide of the present application.

In the present application, the sixth functional module may comprise GSP, D, or a sequence as set forth in SEQ ID NO.21.

In the present application, the sixth functional module may locate at a terminal of the peptide.

In the present application, the order of the first functional module to the fourth functional module may be arbitrary.

For example, the peptide of the present application may comprise the first functional module, the second functional module and the third functional module. For example, the peptide of the present application may consist of the first functional module, the second functional module and the third functional module.

For example, the order of functional modules from the N terminal of the peptide is the first functional module, the second functional module and the third functional module. For example, the order of functional modules from the N terminal of the peptide is the third functional module, the second functional module and the first functional module. For example, the order of functional modules from the N terminal of the peptide is the first functional module, the third functional module and the second functional module. For example, the order of functional modules from the N terminal of the peptide is the third functional module, the first functional module and the second functional module. For example, the order of functional modules from the N terminal of the peptide is the second functional module, the first functional module and the third functional module. For example, the order of functional modules from the N terminal of the peptide is the second functional module, the third functional module and the first functional module.

For example, the peptide of the present application may further comprise the fourth functional module, the fifth functional module, and/or the sixth functional module.

For example, the peptide of the present application may consist of the first functional module, the second functional module, the third functional module, the fourth functional module, and the sixth functional module.

For example, the peptide of the present application may consist of the first functional module, the second functional module, the third functional module, the fourth functional module, the fifth functional module, and the sixth functional module.

For example, the order of functional modules from the N terminal of the peptide is the first functional module, the third functional module, the fourth functional module, the second functional module and the sixth functional module.

For example, the order of functional modules from the N terminal of the peptide is the fifth functional module, the first functional module, the third functional module, the fourth functional module, the second functional module and the sixth functional module.

For example, the order of functional modules from the N terminal of the peptide is the first functional module inserted by at least one (for example, 1 or 2) first functional module, the third functional module, the fourth functional module, the second functional module inserted by at least one (for example, 1 or 2) first functional module, and the sixth functional module inserted by at least one (for example, 1 or 2) the third functional module.

For example, the order of functional modules from the N terminal of the peptide is the fifth functional module, the first functional module inserted by at least one (for example, 1 or 2) first functional module, the third functional module, the fourth functional module, the second functional module inserted by at least one (for example, 1 or 2) third functional module, and the sixth functional module inserted by at least one (for example, 1 or 2) third functional module.

In the present application, the peptide may comprise one or more functional module selected from the first functional module to the sixth functional module.

For example, the number of the first functional module in the peptide of the present application is at least 1, at least 2, at least 3, at least 4, at least 5 or more. For example, the number of the second functional module in the peptide of the present application is at least 1, at least 2, at least 3, at least 4, at least 5 or more. For example, the number of the third functional module in the peptide of the present application is at least 1, at least 2, at least 3, at least 4, at least 5 or more. For example, the number of the fourth functional module in the peptide of the present application is 0, at least 1, at least 2, at least 3, at least 4, at least 5 or more. For example, the number of the fifth functional module in the peptide of the present application is 0, at least 1, at least 2, at least 3, at least 4, at least 5 or more. For example, the number of the sixth functional module in the peptide of the present application is 0, at least 1, at least 2, at least 3, at least 4, at least 5 or more.

For example, the number of the first functional module in the peptide of the present application is 1, the number of the second functional module in the peptide of the present application is 1, the number of the third functional module in the peptide of the present application is 1, the number of the fourth functional module in the peptide of the present application is 1, the number of the fifth functional module in the peptide of the present application is 0, and the number of the sixth functional module in the peptide of the present application is 1.

For example, the number of the first functional module in the peptide of the present application is 1, the number of the second functional module in the peptide of the present application is 1, the number of the third functional module in the peptide of the present application is 1, the number of the fourth functional module in the peptide of the present application is 1, the number of the fifth functional module in the peptide of the present application is 1, and the number of the sixth functional module in the peptide of the present application is 1.

For example, the number of the first functional module in the peptide of the present application is 2, the number of the second functional module in the peptide of the present application is 1, the number of the third functional module in the peptide of the present application is 1, the number of the fourth functional module in the peptide of the present application is 1, the number of the fifth functional module in the peptide of the present application is 1, and the number of the sixth functional module in the peptide of the present application is 1.

For example, the number of the first functional module in the peptide of the present application is 3, the number of the second functional module in the peptide of the present application is 1, the number of the third functional module in the peptide of the present application is 4, the number of the fourth functional module in the peptide of the present application is 1, the number of the fifth functional module in the peptide of the present application is 0, and the number of the sixth functional module in the peptide of the present application is 1.

For example, the number of the first functional module in the peptide of the present application is 3, the number of the second functional module in the peptide of the present application is 1, the number of the third functional module in the peptide of the present application is 4, the number of the fourth functional module in the peptide of the present application is 1, the number of the fifth functional module in the peptide of the present application is 1, and the number of the sixth functional module in the peptide of the present application is 1.

In the present application, the peptide may comprise PKKKRKVG-HHHH (SEQ ID NO:23)-C₁₂-LLHC_{Bzyl}C_{Bzyl}HLL GSPHHD (SEQ ID NO:34), RKKRRQRRR-HHHH (SEQ ID NO:24)-C₁₂-LLHC_{Bzyl}C_{Bzyl}HLL GSPHHD (SEQ ID NO:35), Fmoc-PKKKRKVG (SEQ ID NO:25)-C₁₂-HHHH-C_{stBu}HHC_{stBu}C_{stBu}HHC_{stBu}GSPHHD (SEQ ID NO:36), or Fmoc-C₁₂-PKKKKKVG-HHHH-C_{stBu}HHC_{stBu}C_{stBu}HHC_{stBu}GSPHHD SEQ ID NO:26).

In the present application, the peptide may efficiently bind to the nucleic acids, and/or the peptide may efficiently deliver the nucleic acids into a cell. And the peptide may facilitate the conveyed nucleic acids to escape from the lysis, for example, the lysis caused by an endosome.

### The Selecting Method Thereof

In other aspect, the present application provides a method of selecting a candidate peptide, wherein the method may comprise: Preparing a library of the candidate peptide, wherein the candidate peptide comprises at least two kinds of functional module, and each the functional module is respectively selected from a corresponding library of functional module; wherein the corresponding library of functional module may comprise at least two different functional module sequences.

In the present application, with the benefit of the method, the selection of the candidate peptide is more convenient than ever. Because the selection depends on the combination of various kinds of functional modules of the present application rather than the change of one or more individual amino acids. In the present application, the library of the candidate peptide may comprise various (for example, at least 10², at least 10³, at least 10⁴, at least 10⁵, at least 10⁶, at least 10⁷, at least 10⁸, at least 10⁹, or more) different candidate peptide sequences.

In the present application, each candidate peptide may comprise at least two (for example, at least 2, at least 3, at least 4, at least 5, at least 6, at least 7 or more) different kinds of the functional modules. For example, each candidate peptide may comprise at least two (for example, at least 2, at least 3, at least 4, at least 5, at least 6, at least 7 or more) different kinds of the functional module of the present application.

In the present application, each kind of the functional module which forms the candidate peptide may be originated or selected from a corresponding library of functional module. For example, the first functional module sequence of the present application may be originated or selected from a first functional module library. And in the first functional module library, there may be no kind of functional module sequence other than the first functional module sequence. For example, there may be various different first functional module sequences in the first functional module library. For another example, the second functional module sequence of the present application may be originated or selected from a second functional module library. And in the second functional module library, there may be no kind of functional module sequence other than the second functional module sequence. For example, there may be various different second functional module sequences in the second functional module library.

In the present application, the method may comprise: selecting each functional module sequence respectively from the corresponding library of functional module.

In the present application, the method may comprise: preparing the candidate peptide with the selected different functional modules.

In the present application, the library of functional module may comprise a first functional module library, and the first functional module library may comprise at least two (for example, at least 5, at least 10, at least 10², at least 10³, at least 10⁴, at least 10⁵, at least 10⁶, at least 10⁷, at least 10⁸, at least 10⁹, or more) different first functional module sequences.

In the present application, the first functional module library may comprise at least 10³ (for example, at least 10⁴, at least 10⁵, at least 10⁶, at least 10⁷, at least 10⁸, at least 10⁹, or more) first functional module sequences.

In the present application, the first functional module library may be obtained by chemical synthesis.

In the present application, in the first functional module library, at least 50% (for example, at least 55%, at least 60%, at least 65%, at least 70%, at least 75%, at least 80%, at least 85%, at least 90%, at least 95% or more) of first functional module sequences may comprise at least two (for example, at least 3, at least 4, at least 5, at least 6, at least 7, at least 8, at least 9, at least 10 or more) consecutive lysine.

In the present application, in the first functional module library, at least 30% (for example, at least 35%, at least 40%, at least 45%, at least 50%, at least 55%, at least 60%, at least 65%, at least 70%, at least 75%, at least 80%, at least 85%, at least 90%, at least 95% or more) first functional module sequences may comprise at least three (for example, at least 4, at least 5, at least 6, at least 7, at least 8, at least 9, at least 10 or more) consecutive lysine.

In the present application, the library of functional module may comprise a second functional module library, and the second functional module library may comprise at least two (for example, at least 5, at least 10, at least 10², at least 10³, at least 10⁴, at least 10⁵, at least 10⁶, at least 10⁷, at least 10⁸, at least 10⁹, or more) different second functional module sequences.

In the present application, the second functional module library may comprise at least 10³ (for example, at least 10⁴, at least 10⁵, at least 10⁶, at least 10⁷, at least 10⁸, at least 10⁹, or more) second functional module sequences.

In the present application, the second functional module library may be obtained by chemical synthesis.

In the present application, in the second functional module library, at least 50% (for example, at least 55%, at least 60%, at least 65%, at least 70%, at least 75%, at least 80%, at least 85%, at least 90%, at least 95% or more) second functional module sequence may comprise at least two (for example, at least 3, at least 4, at least 5, at least 6, at least 7, at least 8, at least 9, at least 10 or more) amino acid that comprises a side chain which contains a disulfide bond (for example, a cysteine derivative) and/or at least two amino acids that comprise an imidazole side chain (for example, a histidine) and/or at least two (for example, at least 3, at least 4, at least 5, at least 6, at least 7, at least 8, at least 9, at least 10 or more) non-polar side chain.

In the present application, the library of functional module may comprise a third functional module library, and the third functional module library may comprise at least two (for example, at least 5, at least 10, at least 10², at least 10³, at least 10⁴, at least 10⁵, at least 10⁶, at least 10⁷, at least 10⁸, at least 10⁹, or more) different third functional module sequences.

In the present application, the third functional module library may comprise at least 10³ (for example, at least 10⁴, at least 10⁵, at least 10⁶, at least 10⁷, at least 10⁸, at least 10⁹, or more) third functional module sequence.

In the present application, the third functional module library may be obtained by chemical synthesis.

In the present application, in the third functional module library, at least 50% (for example, at least 55%, at least 60%, at least 65%, at least 70%, at least 75%, at least 80%, at least 85%, at least 90%, at least 95% or more) third functional module sequences may comprise at least two (for example, at least 3, at least 4, at least 5, at least 6, at least 7, at least 8, at least 9, at least 10 or more) consecutive amino acid that comprise an imidazole side chain (for example, a histidine).

In the present application, in the third functional module library, at least 30% (for example, at least 35%, at least 40%, at least 45%, at least 50%, at least 55%, at least 60%, at least 65%, at least 70%, at least 75%, at least 80%, at least 85%, at least 90%, at least 95% or more) third functional module sequence may comprise at least four (for example, at least 5, at least 6, at least 7, at least 8, at least 9, at least 10 or more) consecutive amino acid that comprise an imidazole side chain (for example, a histidine).

In the present application, the library of functional module may comprise a fourth functional module library, and the fourth functional module library may comprise at least two (for example, at least 5, at least 10, at least 10², at least 10³, at least 10⁴, at least 10⁵, at least 10⁶, at least 10⁷, at least 10⁸, at least 10⁹, or more) different fourth functional module sequences.

In the present application, the fourth functional module library may comprise at least 10² (for example, at least 10³, at least 10⁴, at least 10⁵, at least 10⁶, at least 10⁷, at least 10⁸, at least 10⁹, or more) fourth functional module sequences.

In the present application, the fourth functional module library may be obtained by chemical synthesis.

In the present application, in the fourth functional module library, at least 50% (for example, at least 55%, at least 60%, at least 65%, at least 70%, at least 75%, at least 80%, at least 85%, at least 90%, at least 95% or more) fourth functional module sequences may comprise a carbon chain comprising at least three (for example, at least 4, at least 5, at least 6, at least 7, at least 8, at least 9, at least 10 or more) consecutive carbons.

In the present application, in the fourth functional module library, at least 30% (for example, at least 35%, at least 40%, at least 45%, at least 50%, at least 55%, at least 60%, at least 65%, at least 70%, at least 75%, at least 80%, at least 85%, at least 90%, at least 95% or more) fourth functional module sequences may comprise a carbon chain comprising at least ten (for example, at least 11, at least 12, at least 13, at least 14, at least 15, at least 16, at least 17, at least 18, at least 19, at least 20, at least 25 or more) consecutive carbons.

In the present application, the library of functional module may comprise a fifth functional module library, and the fifth functional module library may comprise at least two (for example, at least 5, at least 10, at least 10², at least 10³, at least 10⁴, at least 10⁵, at least 10⁶, at least 10⁷, at least 10⁸, at least 10⁹, or more) different fifth functional module sequences.

In the present application, the fifth functional module library may comprise at least 10² (for example, at least 10³, at least 10⁴, at least 10⁵, at least 10⁶, at least 10⁷, at least 10⁸, at least 10⁹, or more) fifth functional module sequences.

In the present application, the fifth functional module library may be obtained by chemical synthesis. For example, the fifth functional module sequences may be hydrophobic. For example, fifth functional module sequences may comprise an aromatic group. For example, fifth functional module sequences may comprise a Fmoc group.

In the present application, the library of functional module may comprise a sixth functional module library, and the sixth functional module library may comprise at least two (for example, at least 5, at least 10, at least 10², at least 10³, at least 10⁴, at least 10⁵, at least 10⁶, at least 10⁷, at least 10⁸, at least 10⁹, or more) different sixth functional module sequences.

In the present application, the sixth functional module library may comprise at least 10² (for example, at least 10³, at least 10⁴, at least 10⁵, at least 10⁶, at least 10⁷, at least 10⁸, at least 10⁹, or more) sixth functional module sequences.

In the present application, the sixth functional module library may be obtained by chemical synthesis.

In the present application, in the sixth functional module library, at least 90% (for example, at least at least 91%, at least 92%, at least 93%, at least 94%, at least 95%, at least 96%, at least 97%, at least 98%, at least 99% or more) sixth functional module sequences do not form any secondary structure.

In the present application, in the sixth functional module library, at least 99% sixth functional module sequences is hydrophilic.

In the present application, the library of the candidate peptide may comprise at least 10⁵ (for example, at least 10⁶, at least 10⁷, at least 10⁸, at least 10⁹, or more) sequences of the candidate peptide.

In the present application, the library of the candidate peptide may be obtained by chemical synthesis.

In the present application, the candidate peptide may comprise at least one (for example, at least 2, at least 3, at least 4, at least 5, at least 6, at least 7, at least 8, at least 9, at least 10, at least 15, or more) copy of the first functional module.

In the present application, the candidate peptide may comprise at least one (for example, at least 2, at least 3, at least 4, at least 5, at least 6, at least 7, at least 8, at least 9, at least 10, at least 15, or more) copy of the second functional module.

In the present application, the candidate peptide may comprise at least one (for example, at least 2, at least 3, at least 4, at least 5, at least 6, at least 7, at least 8, at least 9, at least 10, at least 15, or more) copy of the third functional module.

In the present application, the candidate peptide may comprise at least one (for example, at least 2, at least 3, at least 4, at least 5, at least 6, at least 7, at least 8, at least 9, at least 10, at least 15, or more) copy of the fourth functional module.

In the present application, the candidate peptide may comprise at least one (for example, at least 2, at least 3, at least 4, at least 5, at least 6, at least 7, at least 8, at least 9, at least 10, at least 15, or more) copy of the fifth functional module.

In the present application, the candidate peptide may comprise at least one (for example, at least 2, at least 3, at least 4, at least 5, at least 6, at least 7, at least 8, at least 9, at least 10, at least 15, or more) copy of the sixth functional module.

In other aspect, the present application provides a peptide, which is prepared by the method of the present application. For example, the peptide of the present application may be prepared by the method of the present application. For example, with the benefit of the method of the present application, the peptide of the present application may be prepared and/or selected.

In other aspect, the present application provides a use of the peptide in preparing a nucleic acid-peptide assembly.

For example, the nucleic acid may comprise a DNA, and/or an RNA. For example, the nucleic acid may comprise a mRNA. For example, the nucleic acid may be single strand and/or double strands.

### Exemplary Polypeptides of the Present Application and Functional Modules Thereof

| ID | NAME | SEQ |
|---|---|---|
| 1 | functional module 1-1 | K |
| 2 | functional module 1-2 | KKK |
| 3 | functional module 1-3 | KKKK (SEQ ID NO: 3) |
| 4 | functional module 1-4 | KKKKK (SEQ ID NO: 4) |
| 5 | functional module 1-5 | PKKKRKVG (SEQ ID NO: 5) |
| 6 | functional module 1-6 | PKKKKKVG (SEQ ID NO: 6) |
| 7 | functional module 1-7 | PRVG (SEQ ID NO: 4) |
| 8 | functional module 2-1 | LLHC_{Bzyl}C_{Bzyl}HLL (SEQ ID NO: 8) |
| 9 | functional module 2-2 | LLC_{Bzyl}C_{BzyI}LL (SEQ ID NO: 9) |
| 10 | functional module 2-3 | C_{stBu}HHC_{stBu}C_{stBu}HHC_{stBu} (SEQ ID NO: 10) |
| 11 | functional module 2-4 | C_{stBu}C_{stBu}C_{stBu}C_{stBu} (SEQ ID NO: 11) |
| 12 | functional module 3-1 | H |
| 13 | functional module 3-2 | HH |
| 14 | functional module 3-3 | HHHH (SEQ ID NO: 14) |
| 15 | functional module 4-1 | 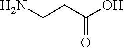 |
| | | (3-aminopropanoic acid, C₃) |
| 16 | functional module 4-2 | 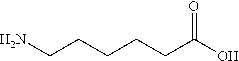 |
| | | (6-aminohexanoic acid, C₆) |
| 17 | functional module 4-3 | 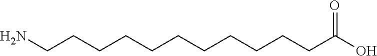 |
| | | (12-aminododecanoic acid, C₁₂) |
| 18 | functional module 4-4 | 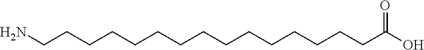 |
| | | (16-aminohexadecanoic acid, C₁₆) |
| 19 | functional module 6-1 | GSP |
| 20 | functional module 6-2 | D |
| 21 | functional module 6-3 | GSPD (SEQ ID NO: 21) |
| 22 | peptide_1 | HHHH-KKKK (SEQ ID NO: 22)-C₁₂- |
| | | LLHC_{Bzyl}C_{Bzyl}HLL GSPD (SEQ ID NO: 33) |
| 23 | peptide_3 | PKKKRKVG-HHHH (SEQ ID NO: 23)-C₁₂- |
| | | LLHC_{Bzyl}C_{Bzyl}HLL GSPHHD (SEQ ID NO: 34) |
| 24 | peptide_5 | RKKRRQRRR-HHHH (SEQ ID NO: 24)-C₁₂- |
| | | LLHC_{Bzyl}C_{Bzyl}HLL GSPHHD (SEQ ID NO: 35) |
| 25 | peptide_7 | Fmoc-PKKKRKVG (SEQ ID NO: 25)-C₁₂-HHHH- |
| | | C_{stBu}HHC_{stBu}C_{stBu}HHC_{stBu}GSPHHD (SEQ ID NO: 36) |
| 26 | peptide_8 | Fmoc-C₁₂-PKKKKKVG-HHHH- - |
| | | C_{stBu}HHC_{stBu}C_{stBu}HHC_{stBu}GSPHHD (SEQ ID NO: 26) |
| 27 | peptide_9 | Fmoc-PKKKRKVG-HHHH- |
| | | C_{stBu}HHC_{stBu}C_{stBu}HHC_{stBu} (SEQ ID NO: 27)-C₁₂- |
| | | GSPHHD (SEQ ID NO: 37) |
| 28 | peptide_10 | Fmoc-C₁₂-PKKKRKVG-HHHH- |
| | | C_{stBu}HHC_{stBu}C_{stBu}HHC_{stBu}-GSPHHD (SEQ ID NO: 28) |
| 29 | peptide_11 | Fmoc-C_{stBu}HHC_{stBu}C_{stBu}HHC_{stBu} (SEQ ID NO: 29)- |
| | | C₁₂-PKKKRKVG-HHHH-GSPHHD (SEQ ID NO: 38) |
| 30 | peptide_12 | Fmoc-PKKKRKVG-HHHH (SEQ ID NO: 30)-C₁₂- |
| | | C_{stBu}HHC_{stBu}C_{stBu}HHC_{stBu}GSPHHD (SEQ ID NO: 39) |
| 31 | peptide_13 | Fmoc-PKKKKKVG-HHHH (SEQ ID NO: 31)-C₁₂- |
| | | C_{stBu}HHC_{stBu}C_{stBu}HHC_{stBu}GSPHHD (SEQ ID NO: 39) |

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1 The Preparation of Peptide-DNA Co-Assemblies

The reported peptide H4 (named as peptide_1 in Table 1) contains an oligohistidine segment (HHHH; SEQ ID NO:14), an oligolysine segment (KKKK; SEQ ID NO:3), a carbon linker segment (C₁₂), a self-assembly segment inserted with histidine (LLHC_{Bzyl}C_{Bzyl}HLL; SEQ ID NO:8), and a hydrophilic segment (GSPD; SEQ ID NO:21). With peptide_1 as a starting point, DNA condensation oligolysine was substituted with dual-functional segments, such as nuclear localization signal peptide (NLS) or cell penetration peptide (TAT), which is switched with N-terminal oligohistidine. In addition, two histidine residues were inserted into the C-terminal hydrophilic segment to form peptide_3 and peptide_5. An aromatic Fmoc group, NLS peptide segment (which also carries positive charges that can condense nucleic acids), histidine residues and redox sensitive t-Butyl-s-s-cysteine (C_{stBu}) were combined together to afford peptide_12 and peptide_13, and the disulfide bond of t-Butyl-s-s-cysteine residues (C_{stBu}) was expected to be reduced in the cellular reducing environment (e.g. in endosome), the histidine residues were expected to be protonated in endosome environment, which in turn lowers the self-assembly ability of the peptides.

TABLE 1
Peptide sequence

| | |
|---|---|
| peptide_1 | HHHH-KKKK (SEQ ID NO: 22)-C₁₂- |
| | LLHC_{Bzyl}C_{Bzyl}HLLGSPD (SEQ ID NO: 33) |
| peptide_3 | PKKKRKVG-HHHH (SEQ ID NO: 23)-C₁₂- |
| | LLHC_{Bzyl}C_{Bzyl}HLLGSPHHD (SEQ ID NO: 34) |
| peptide_5 | RKKRRQRRR-HHHH (SEQ ID NO: 24)-C₁₂- |
| | LLHC_{Bzyl}C_{Bzyl}HLLGSPHHD (SEQ ID NO: 35) |
| peptide_12 | Fmoc-PKKKRKVG-HHHH (SEQ ID NO: 30)- |
| | C₁₂-C_{stBu}HHC_{stBu}C_{stBu}HHC_{stBu}GSPHHD |
| | (SEQ ID NO: 39) |
| peptide_13 | Fmoc-PKKKKKVG-HHHH (SEQ ID NO: 31)- |
| | C₁₂-C_{stBu}HHC_{stBu}C_{stBu}HHC_{stBu}GSPHHD |
| | (SEQ ID NO: 39) |

### Example 2 The Transfection of Peptide-DNA Co-Assemblies

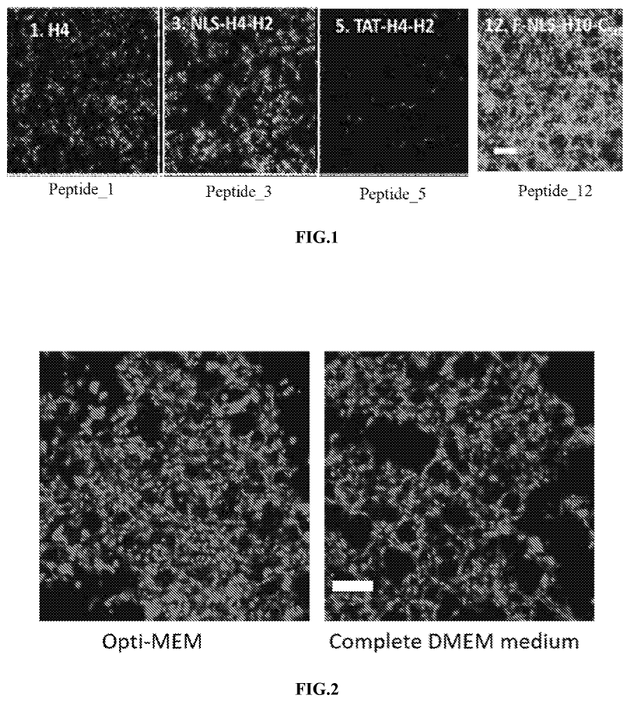

Hek293 cells were used as the model cells. The day before the transfection, Hek293 cells with a density of 0.8×10⁵ cells/well were seeded in a 24-well plate. The next day, the cell medium (0.35 ml) was either replaced with Opti-MEM (0.35 ml) of low serum. Then, 3 μl freshly prepared samples were added (the final DNA concentration in the medium depends on the DNA concentration in the freshly prepared stocks). After 1-day and 2-day incubation at 37° C. in the presence of 5% CO2-, the green transfected cells (represented by the white part in FIG. 1.) were imaged and recorded under the fluorescence microscope with the magnification of ×10.

### Example 3 The Influence of Medium on the Transgene Expression for Peptide_12

Two parallel experiments were investigated in Opti-MEM with 0.5% serum and complete Dulbecco's modified Eagle's medium (DMEM) with 10% fetal bovine serum. In Opti-MEM with medium replacement and in complete DMEM without medium replacement, similar density of transfected cells was obtained (FIG. 2). This suggested that serum had minimal effect on the biological function of peptide_12-DNA co-assemblies, which implies its potential for in vivo application.

### Example 4 The Optimization of Component (Peptide and DNA) Concentration in DMEM for Peptide_12

### (1) The Effect of DNA Concentration Based on Peptide Concentration at 0.5 mM

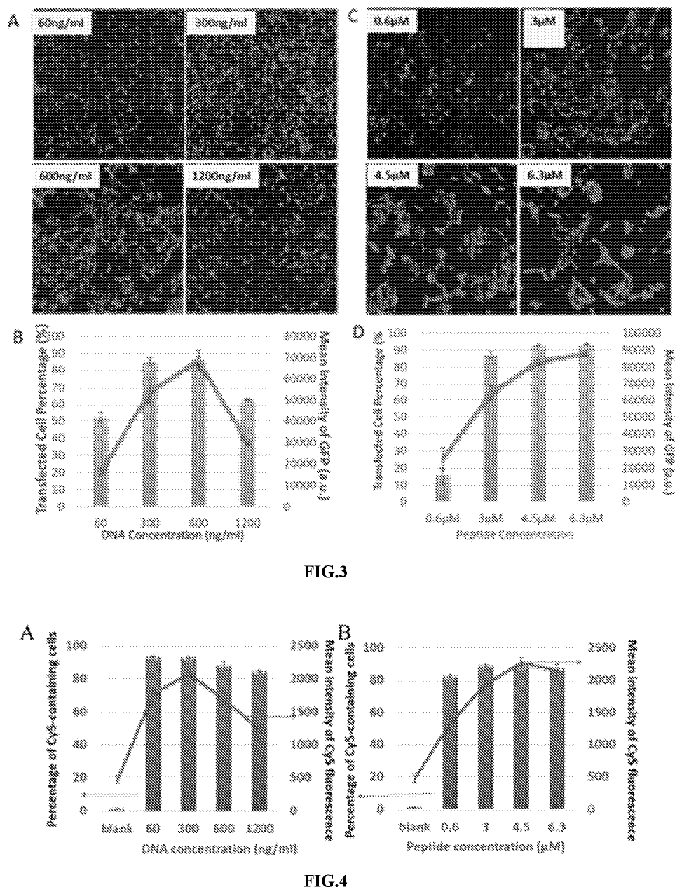

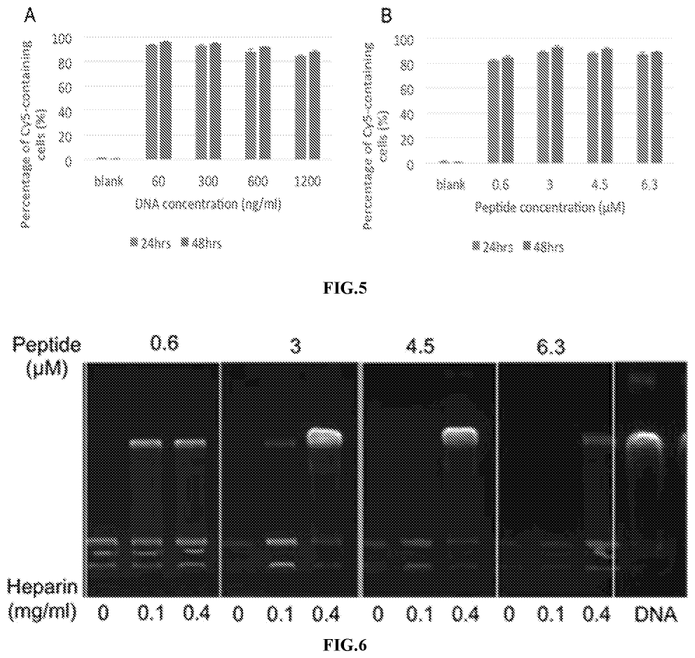

The effect of DNA concentration was optimized through fixing the final peptide concentration (3 μM) and varying DNA amount (from 30 ng/well, 150 ng/well, 300 ng/well to 600 ng/well) in cell medium. One-day incubation led to the observation of green cells (data not shown) and 2-day incubation resulted in the significant enhancement of transgene expression (FIG. 3A). When DNA amount was up to 600 ug/well, decrease of green cell density was observed (FIG. 3A). The FACS analysis showed the consistent trend as that by fluorescence microscopy imaging. At DNA concentration of 60 ng/ml, the percentage of transfected cells reached to 56%. Increasing of DNA concentration to 300 ng/ml and 600 ng/ml significantly enhanced the transfection efficiency over 85%; while further increase the DNA up to 1200 ng/ml led to the transfection efficiency drop down to 63% (FIG. 3B). With DNA concentration increasing, the mean fluorescence intensity of GFP reaches to the peak at DNA concentration of 600 ng/ml, followed by drop down. This suggested the intermediate DNA of 600 ng/ml was the optimal DNA concentration. This suggested that the same amount of peptide (3 μM) could efficiently co-assemble with DNA with the concentration up to 600 ng/ml; while at DNA of 1200 ng/ml, excess DNA may interrupt the co-assembling process, compromising the transgene expression.

(2) The Effect of Peptide Concentration Based on DNA Concentration at 100 μg/ml

The peptide concentration through keeping DNA at 600 ng/ml and varying peptide from 0.6 μM, 3 μM, 4.5 μM and 6.3 μM in co-assembly samples. One-day incubation led to obvious transfection (data not shown) and 2-day incubation resulted in 15% transfected cells with peptide concentration as low as 0.6 μM; while increase of the peptide concentration to 3 μM, 4.5 μM and 6.3 μM significantly enhanced the transfection efficiency to around 90% (FIG. 3C,3D). Though mean fluorescence intensity of GFP increased at peptide concentration of 4.5 μM and 6.3 μM (FIG. 3D), obvious cell agglomeration was observed under both peptide concentrations, so the peptide concentration of 3 μM was chosen in the following studies.

### (3) The Mechanism of the Concentration-Dependent Transgene Expression

To understand the underlying mechanism of the concentration-dependent transgene expression, the DNA delivery capability of the co-assemblies was evaluated with Cy5-labeled DNA as the probe. With the fixed peptide concentration of 3 μM and varied DNA concentration from 60 ng/ml to 1200 ng/ml in cell medium, FACS analysis demonstrated that 24 hr-incubation led to the DNA internalization efficiency reached over 90%, which was negligible on the varied DNA concentration (FIG. 4A). Elongation of the incubation time from 24 hrs to 48 hrs led to the slight enhancement of Cy5-DNA cell population (FIG. 5A). However, the mean fluorescence intensity of Cy5 reached the peak at DNA concentration of 300 ng/ml, and significant drop was observed at DNA concentration of 1200 ng/ml. This trend was consistent with the mode of mean fluorescence intensity observed in FIGS. 3A-3B, which explained the DNA concentration-dependent transgene expression efficiency.

Fixed DNA concentration of 600 ng/ml and varied peptide concentration from 0.6 μM to 6.3 μM in cell culture medium, the result was that 24-hr incubation resulted in 86±4% of the cellular internalization efficiency (FIG. 4B), and minimal enhancement was observed after 48 hr-incubation (FIG. 5B). The mean fluorescence intensity of Cy5 gradually increased and reached the plateau at peptide concentration of 4.5 μM. This suggested that the internalized amount of DNA was regulated by peptide concentration, which eventually influenced the transgene expression of delivered GFP-encoded plasmid.

### Example 5 The Stability of Peptide-DNA Co-Assemblies

By gel electrophoresis assay (FIG. 6), all the co-assemblies were stable without DNA movement upwards. In the presence of heparin of 0.1 mg/ml, the co-assemblies with low peptide concentration (0.6 μM) started to release DNA. With high concentration of heparin (0.4 mg/ml), the co-assemblies with intermediate peptide concentration (3 and 4.5 μM) both had DNA released out, however, co-assemblies with peptide of 6.3 μM only showed minimal DNA release. This indicated that peptide concentration within these co-assembles with fixed DNA concentration regulated the stability and DNA release capability. The balanced particle stability/DNA release capability with intermediate peptide concentration was critical to achieve high transgene expression.

### Example 6 The Preparation of Peptide-mRNA Co-Assemblies

Peptide_12 was dissolved in DMSO at 25 mM or 10 mM as stock in room temperature. The stock HEPES (100 mM, pH 9) buffer was added into the MQ water containing a model 5-Methoxy-U modified mRNA expressing GFP protein. The sequence of the mRNA was SEQ ID NO.32. The peptide stock solution was added into the buffered solution followed by vortex mixing for 5 seconds and incubated under room temperature for 30 min. Table 2 showed different Peptide-mRNA co-assemblies under different N/P ratio.

The sequence of the mRNA is as follow:

(SEQ ID NO: 32)
AUGGUGAGCAAGGGCGAGGAGCUGUUCACCGGGGUGGUGCCCAUCCUGG
UCGAGCUGGACGGCGACGUAAACGGCCACAAGUUCAGCGUGUCCGGCGA
GGGCGAGGGCGAUGCCACCUACGGCAAGCUGACCCUGAAGUUCAUCUGC
ACCACCGGCAAGCUGCCCGUGCCCUGGCCCACCCUCGUGACCACCCUGA
CCUACGGCGUGCAGUGCUUCAGCCGCUACCCCGACCACAUGAAGCAGCA
CGACUUCUUCAAGUCCGCCAUGCCCGAAGGCUACGUCCAGGAGCGCACC
AUCUUCUUCAAGGACGACGGCAACUACAAGACCCGCGCCGAGGUGAAGU
UCGAGGGCGACACCCUGGUGAACCGCAUCGAGCUGAAGGGCAUCGACUU
CAAGGAGGACGGCAACAUCCUGGGGCACAAGCUGGAGUACAACUACAAC
AGCCACAACGUCUAUAUCAUGGCCGACAAGCAGAAGAACGGCAUCAAGG
UGAACUUCAAGAUCCGCCACAACAUCGAGGACGGCAGCGUGCAGCUCGC
CGACCACUACCAGCAGAACACCCCCAUCGGCGACGGCCCCGUGCUGCUG
CCCGACAACCACUACCUGAGCACCCAGUCCGCCCUGAGCAAAGACCCCA
ACGAGAAGCGCGAUCACAUGGUCCUGCUGGAGUUCGUGACCGCCGCCGG
GAUCACUCUCGGCAUGGACGAGCUGUACAAGUAA

TABLE 2
The peptide-mRNA co-assemblies of different N/P ratio

| mRNA | 100 μg/ml (1 μl mRNA stock in every 10 μl sample solution) | | | | | | | | | |
|---|---|---|---|---|---|---|---|---|---|---|
| N/P | 0.8 | | 2 | | 4 | | 8 | | 16 | |
| Peptide | 0.052 | mM | 0.130 | mM | 0.26 | mM | 0.5 | mM | 1 | mM |
| w/w | 2:1 | | 5:1 | | 10:1 | | 20:1 | | 40:1 | |
| MQ water | 8.25 | μl | 8 | μl | 7.5 | μl | 7.5 | μl | 6 | μl |
| HEPES | 0.75 | μl | 1 | μl | 1.5 | μl | 1.5 | μl | 3 | μl |

### Example 7 The Transgene Expression of Peptide-mRNA Co-Assemblies

### (1) The Transfection Condition Optimization in Hela Cell

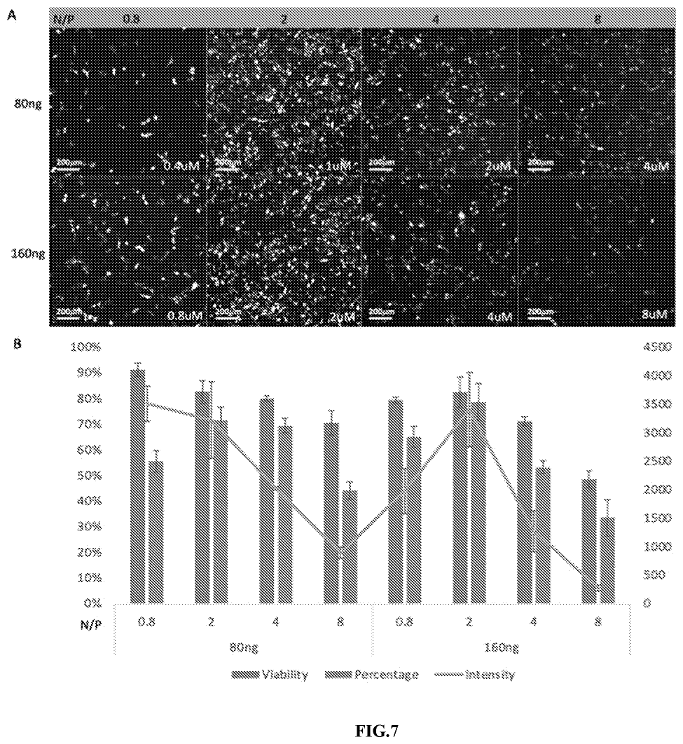

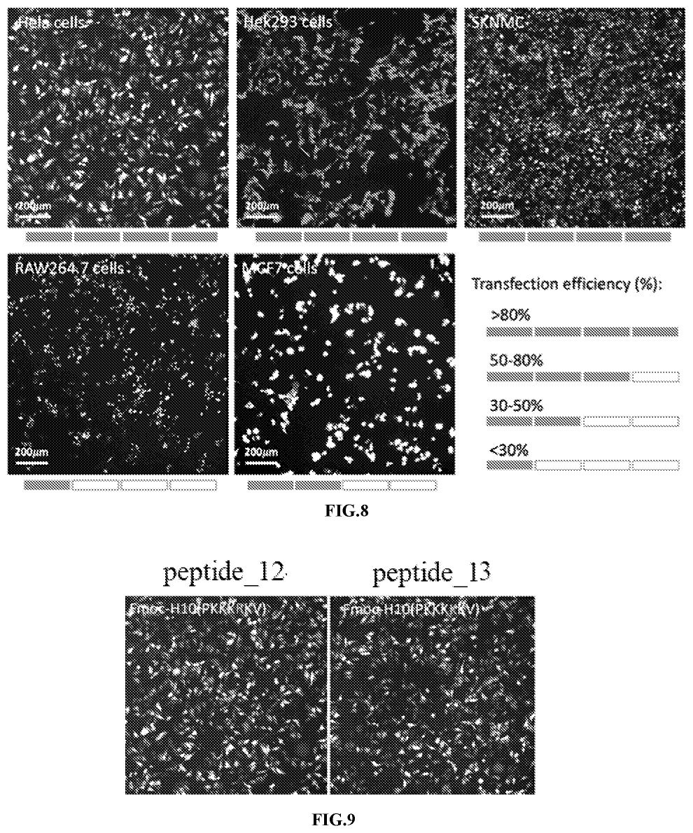

FIG. 7A showed the fluorescent microscope images of Hela cells, following 24 hours transfection with the peptide-mRNA co-assemblies. The transfection conditions were optimized with N/P ratio from 0.8, 2, 4, 8 with mRNA concentrations at 80 ng/well and 160 ng/well (96-well plate). FIG. 7B showed the quantification of the transfected cells with flow cytometry. The transfection efficiency (number of cells expressing GFP/total number of cells) and the intensity of the fluorescent was quantified with flow cytometry on a FACS Aria III flow cytometer. Quantification was carried out with the cell viability, transfected cell percentage and the mean fluorescent intensity of the transfected cells. The result showed that the cell viability and transfected percentage were high with N/P of 2.

### (2) The Transfection Efficiency of Peptide-mRNA Co-Assemblies in Different Cell Lines.

The optimized mRNA and peptide concentration were different for different cell lines, such as 100 ng/well mRNA and 5 μM peptide for Hek293 cells; 160 ng/well mRNA and 2 μM peptide for Hela cells; 80 ng/well mRNA and 1 μM peptide for SKNMC cells; 380 ng/well mRNA and 5 μM peptide for RAW264.7 cells; 160 ng/well mRNA and 4 μM peptide for MCF7. The transfection condition for each cell line was optimized and the best transfection condition was selected as the cell viability was above 80%. The results were shown in FIG. 8. The percentage of transfected Hek293 cells, Hela cells and SKNMC cells was above 80%.

### Example 8 The Transfection Efficiency of Peptide-mRNA Co-Assemblies After Freeze-Drying

### (1) The Preparation of the Peptide-mRNAA Co-Assemblies

The day before the transfection, Hela cells were seeded with a density of 1.5×10⁴ cells/well in a 96-well plate; 24 hours later, the cell confluence reached 80-90%. The cells of GFP-expression were imaged under the fluorescence microscope with ×10 objective lens. Transfection conditions were also optimized for other cell lines. Hek293 cells, SKNMC cells, RAW264.7 cells and MCF7 cells were seeded one day before the transfection at 1-2×10⁴ cells/well to reach the optimal confluence in 96-well plate. Peptide-mRNA co-assemblies with different N/P ratio from 2 to 8 were tested in different cell lines. The transfection conditions were settled according to the highest transfected cell percentage with above 80% cell viability and the lowest required mRNA amount.

The sample solution was mixed with the cryoprotectant (sucrose solution) at 1:1 ratio to obtain the final desired concentration of the sucrose at 5, 10 or 20% (w/v). Then, the peptide-mRNA co-assemblies were quickly frozen in liquid nitrogen, followed by drying on a freeze-dryer. The peptide-mRNA co-assemblies were reconstituted with MQ water to its original concentration and further evaluated with its transfection efficiency.

### (2) The Transfection Efficiency of Peptide_12-mRNA Co-Assemblies vs Peptide_13-mRNA Co-Assemblies

Hela cells were transfected with the peptide_12-mRNA co-assemblies and peptide_13-mRNA co-assemblies. The fluorescence images shown in FIG. 9 displayed the similar percentage of transfection green cells, suggesting similar transfection capability for both peptide-mRNA co-assemblies.

### Example 9 Formation of Peptide_13-mRNA Co-Assemblies

We mixed an increasing amount of peptide with a fixed amount of mRNA, where the peptide amount to mRNA is indicated by the N/P ratio, the molar ratio between the positively charged nitrogen (NH3+) in peptide and negatively charged phosphate (PO4−) in mRNA backbone. The morphologies of the co-assemblies weremonitored by TEM and the nanoparticles' hydrodynamic distributions were measured by Dynamic Light Scattering (DLS).

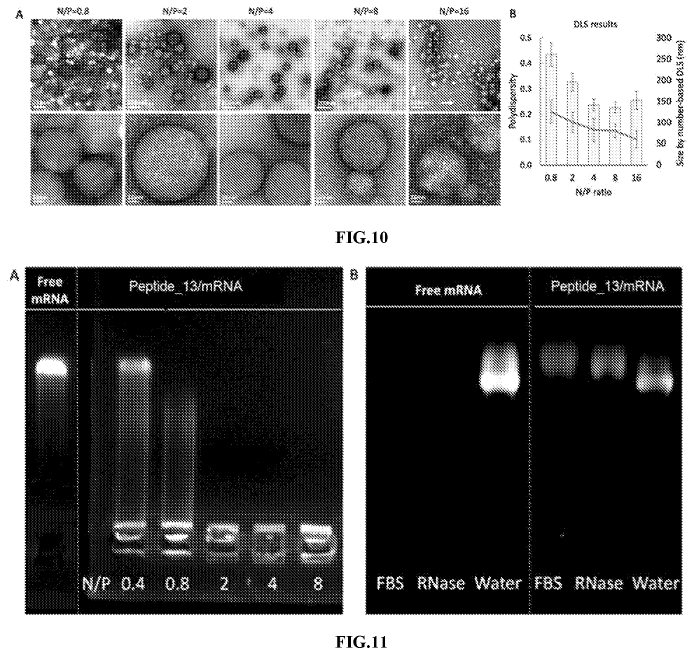

From the TEM image, at N/P ratio of 0.8, the peptide and mRNA formed a network-like structure. The magnified TEM pictures showed that under this N/P ratio, most of the nanoparticles adhered to each other. When the N/P ratio was increased to 2, dispersed nanoparticles as large as approximately 130±60 nm by TEM images (>50 measurement) and 200±20 nm by DLS were observed, with larger hydrodynamic sizes conventionally acceptable, indicating successful nanoparticles formation at N/P=2. When N/P ratio was increased to 4 until 16, the size of nanoparticles decreased to approximately 100±40 nm by TEM and 140±30 nm by DLS. Meanwhile, starting from N/P=8, small fibril-like structures at the background could be observed, which might be the excessive peptide self-assemblies (FIG. 10A).

### Stability of Peptide_13-mRNA Co-Assemblies by RNase

Agarose gel electrophoresis for the stability of peptide_13-mRNA co-assemblies: The stability of the co-assemblies at different N/P ratios was evaluated with agarose gel electrophoresis retardation assay. The co-assemblies at different N/P ratios were prepared 30 minutes before electrophoresis on gel. 1% agarose gel was prepared, and electrophoresis was carried out in 1×TBE buffer at 150V for 15 minutes. The gel was stained by SYBRTM Gold Nucleic Acid Gel Stain (#S11494) for 8 minutes and results were visualized under an ultraviolet transilluminator. The degradation challenge test was carried out by incubating the co-assemblies with serum or RNase and analyzed by agarose gel electrophoresis. Free mRNA was prepared and treated at the same conditions as the co-assemblies as control. To check for enzyme stability, co-assemblies and controls were first incubated in 50 ng/μl RNase A for 30 minutes at 37° C. RNase A was then degraded by RNAsecureTM RNase Inactivation Reagent (catalog #AM7005) based on the protocol. For serum stability, particles were incubated in Fetal Bovine Serum (FBS) for 30 minutes at 37° C. 1 mg/ml heparin (heparin/mRNA=10:1, w/w) were added and allowed to incubate for 30 minutes to destabilize the co-assemblies and displace mRNA inside. Agarose gel electrophoresis was carried out at 150V for 15 minutes followed by SYBRTM Gold stain and ultraviolet transilluminator observation.

The stability of peptide_13-mRNA co-assemblies under different N/P ratios was estimated with agarose gel electrophoresis assay. (FIG. 11A).

As shown, mRNA disassociated from the co-assemblies at N/P=0.4 and a smeared band was observed at N/P=0.8. Starting from N/P=2, mRNA remained retarded within the co-assemblies. This indicated peptide_13-mRNA co-assembled with mRNA into a stabled structure when increasing N/P ratio to 2. This ratio was used as a reference for later cell transfection optimization. Based on this, we continued to investigate the protective effect of mRNA by peptide_13-mRNA against serum and enzymatic degradation at N/P=2. (FIG. 11B) After the degradation challenge, the mRNA was completely dissociated from co-assemblies by adding heparin at 10:1 heparin/mRNA (w/w) ratio. Here, mRNA band was absent from the free mRNA samples co-incubated with serum or RNase, indicating mRNA had been completely digested. With peptide_13-mRNA, blurred mRNA bands were observed with a slightly upper shift, indicating that mRNA remained despite partial degradation. This suggested that the co-assemblies could protect mRNA from digestion by RNase A and serum.

### Biocompatibility of Peptide_13

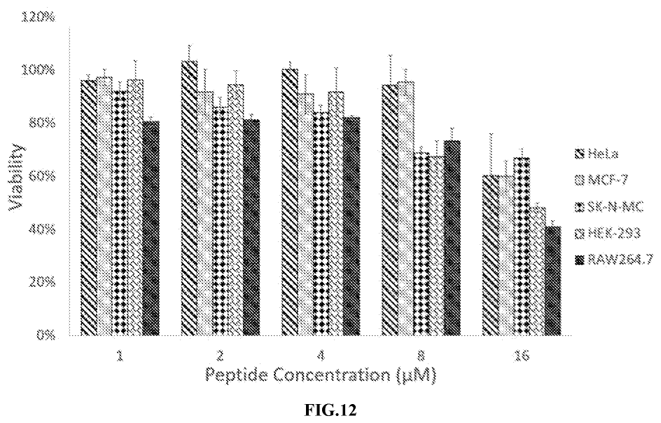

MTT assay was applied to evaluate the cytotoxicity effect of the peptide on HeLa, MCF-7, SK-N-MC, HEK293 and RAW264.7 cells (FIG. 12).

In HeLa and MCF-7 culture, cell viability was above 80% at peptide_13 concentration up to 8 μM. In SK-N-MC, HEK293 and RAW264.7 culture, cell viability was above 80% at peptide concentration up to 4 μM. Therefore, for subsequent transfection optimization experiments, peptide concentrations were kept up to 8 μM maximum for HeLa and MCF-7 cells, and up to 4 μM maximum for SK-N-MC, HEK293 and RAW264.7 cells.

### Cytocompatibility and Transfection Efficiency of Peptide_13-mRNA Co-Assemblies in HeLa Cells

The transfection efficiency was measured via fluorescence microscopy and quantified by flow cytometry analysis.

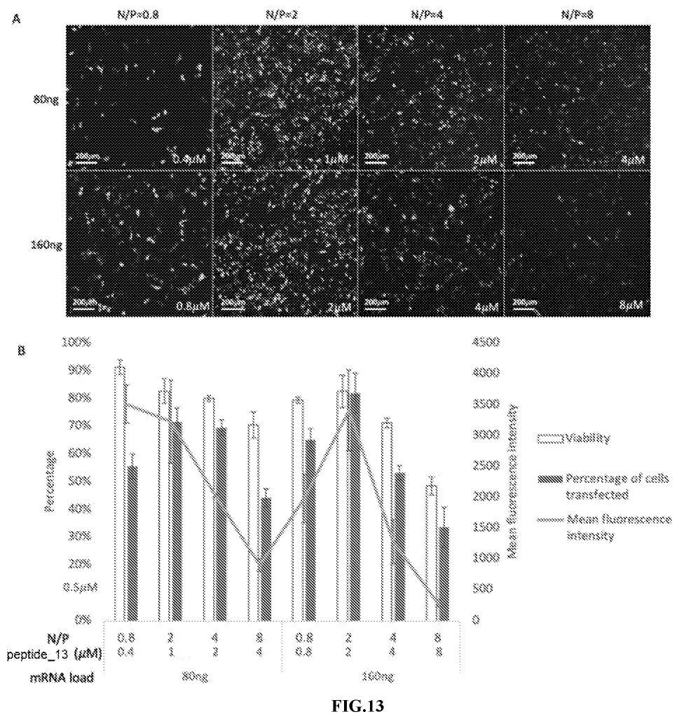

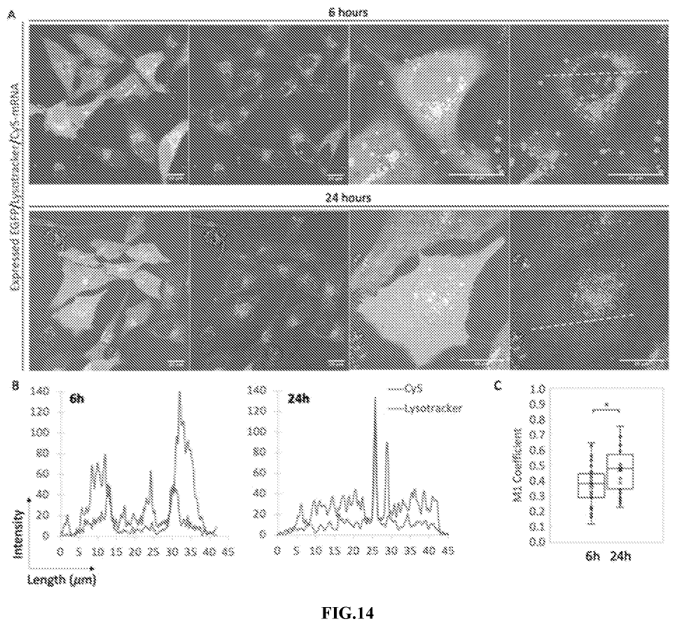

We evaluated transfection by fixing the mRNA amount at 80 ng/well or 160 ng/well while altering the peptide concentration from 0.4 μM to 8 μM, corresponding to N/P ratio of 0.8, 2, 4, 8. (FIG. 13A-13B). Co-assemblies began to show cytotoxicity at 4 μM, with cell viability dropped down to 70%. For peptide concentration lower than 4 μM, cell viability was all above 80%, which include N/P=0.8, 2, 4 for 80 ng/well mRNA and N/P=0.8, 2 for 160 ng/well mRNA. The optimal peptide concentration for 80 ng/well and 160 ng/well mRNA was 1 μM (N/P=2) and 2 μM (N/P=2) respectively, which achieved 73% and 80% transfected cells percentage and comparatively high mean fluorescence intensity.

Successful endosomal escape of mRNA is a prerequisite for efficient transfection. The intracellular trafficking of the peptide_13-mRNA co-assembly was observed by live-cell confocal microscope (FIG. 14A) and quantified both the line profiles (FIG. 14B) and the Mander's overlap coefficient M130 (FIG. 14C).

We tracked the Cy5-labeled mRNA in HeLa cells and labeled the late endosome/lysosome with Lysotracker Red. After 6-hour incubation followed by washing, some cells started producing EGFP. Within these high-expressing cells, mRNA was evenly distributed inside the cells' cytosol, indicating efficient endosomal escaping. After 24 hours, most of the cells were EGFP positive while no or very faint mRNA could be observed inside the cytosol, indicating mRNA undergoes fast degradation inside the cytosol after being translated. This mRNA decrease could also be observed in the intensity line profiles extracted from the enlarged confocal image. ((FIG. 14B) The magenta color indicated the colocalization of the mRNA with the late endosome/lysosome. We quantified this colocalization with the Mander's overlap coefficient M1 (0<M1<1), corresponding to the fraction of mRNA colocalized with late endosome/lysosomes. ((FIG. 14C). Thus, a lower M1 value indicated greater endosomal escape of mRNA. Both M1 values (6 and 24 hours) below 0.5 suggested weak colocalization. The results suggested that mRNA had achieved successfully endosomal escape.

### Transfection Efficiency of Peptide_13-mRNA Co-Assemblies in Other Cell Lines.

Transfection efficiency was also studied in other cell lines, including MCF-7, SKNMC, RAW264.7 and HEK293. Optimal transfection conditions were chosen according to the transfected cells percentage and mean fluorescence intensity, with cell viability above 80%. For MCF-7 cells, the mRNA concentration was varied from 80 ng/well to 240 ng/well, and N/P ratio was varied from 0.8 to 8. The optimal transfection condition for MCF-7 was 160 ng/well mRNA with N/P at 4. At this condition, 43% of the cells were transfected with the highest mean fluorescence intensity. Co-assemblies with lower N/P ratios (0.8 and 2) showed low transfection efficiency from 21% and 35%. Further increasing the N/P to 8 resulted in lower fluorescence intensity and unacceptable cytotoxicity to cells, which aligned with our MTT results. As for the SKNMC cells, preliminary trial with coassemblies at N/P=0.8 showed minimum transfection effects, whereas co-assemblies with N/P=8 showed unacceptable cytotoxicity and low fluorescence intensity, as expected. In the second trial, the mRNA concentration was varied at 80 ng/well and 160 ng/well, and N/P ratio was varied at 2, 3 and 4. The optimal transfection condition for SK-N-MC was 160 ng/well mRNA with N/P at 2, which yielded a transfected cell percentage above 90% and comparable fluorescence intensity as LipoMMAX. For RAW264.7 cells, the mRNA concentration was varied from 20 ng/well to 400 ng/well, and N/P ratio was varied from 2 to 16. The optimal transfection condition for RAW264.7 was at 380 ng/well mRNA with N/P at 2, where the transfected cell percentage reached 20% with the highest mean fluorescence intensity. While at 50 ng/well mRNA and an N/P at 16, the co-assemblies also achieved 20% of transfected cells, albeit at a relatively low intensity. For HEK293 cells, the mRNA concentration was varied at 10 ng/well and 150 ng/well, and N/P ratio was varied from 2 to 16. The optimal transfection condition for HEK293 cells was 100 ng/well mRNA with N/P at 8, where 83% cells were transfected with the highest mean fluorescence intensity.

The transfection efficiencies of the co-assemblies under optimal conditions in various cell lines were summarized in FIG. 15, with detailed transfection conditions and efficacy comparison with LipoMMAX. We obtained intriguing results that the optimal transfection conditions for different cell lines were diverse. Despite of different mRNA loading amount, the optimal transfection condition for HeLa, SKNMC and RAW264.7 cells were at N/P=2. For MCF7 cells and HEK293 cells the optimal N/P ratio were 4 and 8 respectively. As previously described, RAW264.7 cells also achieved a similar percentage of transfected cells at N/P=16 as at N/P=2.

### Stability of Peptide_13-mRNA Co-Assemblies After Lyophilization

To evaluate the suitability of the new peptide-based carriers for transportation and long-term storage, different conditions to preserve the peptide_13-mRNA co-assemblies were explored. Based on previous experiments, the co-assemblies with N/P ratio at 2 and mRNA concentration of 160 ng/well on HeLa cells were selected for testing.

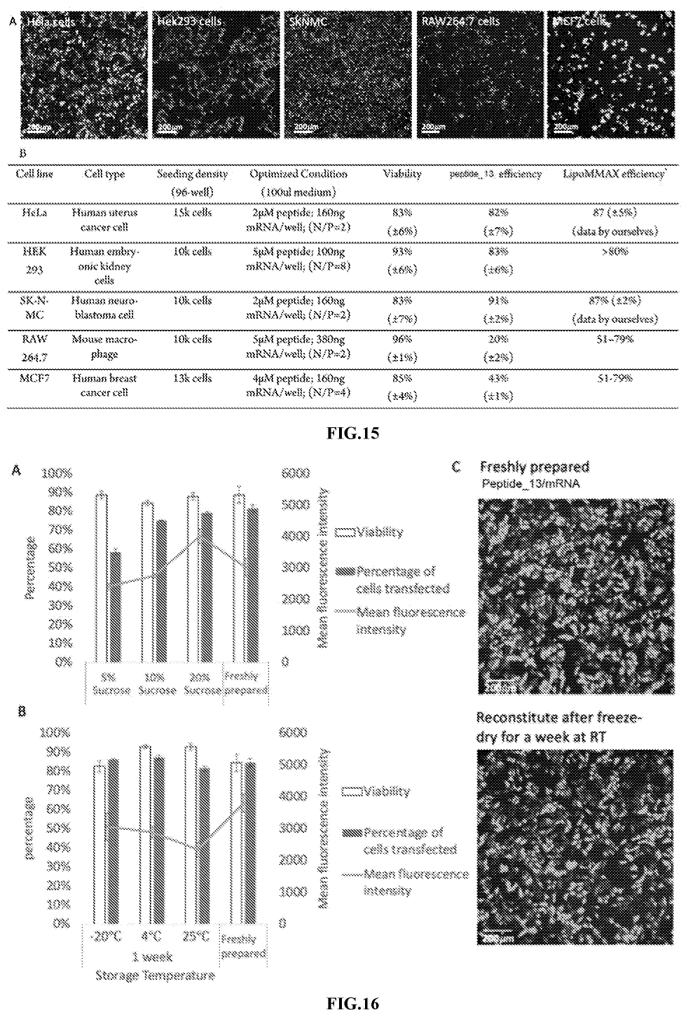

For lyophilization, the sample solution was mixed with the cryoprotectant (sucrose solution) at a 1:1 (v/v) to obtain the final desired concentration of the sucrose at 5, 10 or 20% (w/v). Then, the co-assemblies were quickly frozen in liquid nitrogen and freezedried at −100° C. and 0.003 mbar. After the co-assemblies were dried, they were reconstituted with MQ water to their original concentration. Transfection and quantification were similar to previous examples. Referring to FIG. 16A, co-assemblies freeze-dried with 5%, 10%, 20% sucrose (w/v) were able to reach transfection efficiency of 58%, 75% and 80% respectively, compared to freshly prepared co-assemblies with 82% transfection. Thus, lyophilization in the presence of 20% sucrose can help preserve transfection functionality of the delivery system up to 97%.

Co-assemblies lyophilized at 20% sucrose (w/v) were selected to test for mRNA integrity at different storing temperatures. Samples were lyophilized as previous example, and stored in −20° C., 4° C., and room temperature (25° C.) for a week. Transfection and quantification were repeated as previous examples.

FIG. 16B shows that, upon reconstitution, peptide_13-mRNA co-assemblies attained transfection of 86%, 87% and 82% respectively in HeLa cells with viability over 85%. In summary, both transfection efficiency and cell viability were maintained after storage.

While preferred embodiments of the present invention have been shown and described herein, it will be obvious to those skilled in the art that such embodiments are provided by way of example only. It was not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-141. (canceled)

142. A peptide, wherein said peptide comprises a first functional module, a second functional module and a third functional module, wherein said first functional module is able to bind to a nucleic acid, said second functional module is able to self-assemble outside the cell and disassemble inside the cell, and said third functional module is able to be protonated in endosome, wherein the peptide is able to form an assembly with nucleic acid.

143. The peptide of claim 142, wherein said first functional module comprises one or more lysine and/or arginine.

144. The peptide of claim 142, wherein said first functional module comprises K, KKK, or a sequence as set forth in any one of SEQ ID NOs:3-7.

145. The peptide of claim 142, wherein said second functional module comprises a polypeptide for which the self-assembly propensity is able to be tuned by an intracellular or external stimuli.

146. The peptide of claim 142, wherein said second functional module is charged, and/or is less hydrophobic and/or is able to disassemble the beta sheets after encountering said intracellular stimuli.

147. The peptide claim 142, wherein said second functional module comprises at least one amino acid that comprises a side chain which contains a disulfide bond.

148. The peptide of claim 142, wherein said second functional module comprises at least one amino acid that comprises an imidazole side chain.

149. The peptide of claim 142, wherein said second functional module comprises at least one amino acid that comprises a non-polar side chain.

150. The peptide of claim 142, wherein said second functional module comprises one or more alanine, asparagine, cysteine, glutamine, histidine, isoleucine, leucine, methionine, phenylalanine, serine, threonine, tryptophan tyrosine, valine, S-Benzyl-L-cysteine (C_{bzyl}), t-butyl-s-s-cysteine (C_{stBu}) and/or the combination thereof.

151. The peptide of claim 142, wherein said second functional module comprises a sequence as set forth in any one of SEQ ID NOs:8-11.

152. The peptide of claim 142, wherein said third functional module comprises at least one amino acid comprising an imidazole side chain.

153. The peptide of claim 142, wherein said third functional module comprises one or more copies of Histidine (H).

154. The peptide of claim 142, wherein said third functional module comprises H, HH, or a sequence as set forth in SEQ ID NO:14.

155. The peptide of claim 142, wherein said peptide comprises a fourth functional module, and said fourth functional module comprises a linker.

156. The peptide of claim 155, wherein said fourth functional module comprises 3-aminopropanoic acid (C₃), 6-aminohexanoic acid (C₆), 12-aminododecanoic acid (C₁₂), or 16-aminohexadecanoic acid (C₁₆).

157. The peptide of claim 142, wherein said peptide comprises a fifth functional module, and said fifth functional module comprises a hydrophobic end moiety.

158. The peptide of claim 142, wherein said peptide comprises a sixth functional module, and said sixth functional module is hydrophilic.

159. The peptide of claim 142, wherein said sixth functional module comprises GSP, D, or a sequence as set forth in SEQ ID NO:21.

160. The peptide of claim 142, wherein said peptide comprises PKKKRKVG-HHHH (SEQ ID NO:23)-C₁₂-LLHC_{Bzyl}C_{Bzyl}HLL GSPHHD (SEQ ID NO:34), RKKRRQRRR-HHHH (SEQ ID NO:24)-C₁₂-LLHC_{Bzyl}C_{Bzyl}HLL GSPHHD (SEQ ID NO:35), Fmoc-PKKKRKVG (SEQ ID NO:25)-C₁₂-HHHH-C_{stBu}HHC_{stBu}C_{stBu}HHC_{stBu}GSPHHD (SEQ ID NO:36), or Fmoc-C₁₂-PKKKKKVG-HHHH-C_{stBu}HHC_{stBu}C_{stBu}HHC_{stBu}GSPHHD (SEQ ID NO:26).

161. A method of selecting a candidate peptide, wherein said method comprises:
preparing a library of said candidate peptide, wherein said candidate peptide comprises at least two kinds of functional module, and each said functional module is respectively selected from a corresponding library of functional module;
wherein said corresponding library of functional module comprises at least two different functional modules sequences.


SEQUENCE LISTING (40 sequences)

SEQ ID NO: 1-2 - skipped (2 entries)

SEQ ID NO: 3 - protein, 4 aa, synthetic construct
KKKK

SEQ ID NO: 4 - protein, 5 aa, synthetic construct
KKKKK

SEQ ID NO: 5 - protein, 8 aa, synthetic construct
PKKKRKVG

SEQ ID NO: 6 - protein, 8 aa, synthetic construct
PKKKKKVG

SEQ ID NO: 7 - protein, 4 aa, synthetic construct
PRVG

SEQ ID NO: 8 - protein, 8 aa, synthetic construct
  MOD_RES 4..5: note = S-Benzyl-L-cysteine
LLHCCHLL

SEQ ID NO: 9 - protein, 6 aa, synthetic construct
  MOD_RES 3..4: note = S-Benzyl-L-cysteine
LLCCLL

SEQ ID NO: 10 - protein, 8 aa, synthetic construct
  MOD_RES 1: note = s-(tert-butylthio)-L-cysteine
  MOD_RES 4..5: note = s-(tert-butylthio)-L-cysteine
  MOD_RES 8: note = s-(tert-butylthio)-L-cysteine
CHHCCHHC

SEQ ID NO: 11 - protein, 4 aa, synthetic construct
  MOD_RES 1..4: note = s-(tert-butylthio)-L-cysteine
CCCC

SEQ ID NO: 12-13 - skipped (2 entries)

SEQ ID NO: 14 - protein, 4 aa, synthetic construct
HHHH

SEQ ID NO: 15-20 - skipped (6 entries)

SEQ ID NO: 21 - protein, 4 aa, synthetic construct
GSPD

SEQ ID NO: 22 - protein, 8 aa, synthetic construct
  LIPID 8: note = 12-aminododecanoic acid
HHHHKKKK

SEQ ID NO: 23 - protein, 12 aa, synthetic construct
  LIPID 12: note = 12-aminododecanoic acid
PKKKRKVGHHHH

SEQ ID NO: 24 - protein, 13 aa, synthetic construct
  LIPID 13: note = 12-aminododecanoic acid
RKKRRQRRRHHHH

SEQ ID NO: 25 - protein, 8 aa, synthetic construct
  SITE 1: note = fluorenyl methoxycarbonyl-Proline
  LIPID 8: note = 12-aminododecanoic acid
PKKKRKVG

SEQ ID NO: 26 - protein, 27 aa, synthetic construct
  LIPID 1: note = 12-aminododecanoic acid
  MOD_RES 14: note = s-(tert-butylthio)-L-cysteine
  MOD_RES 17..18: note = s-(tert-butylthio)-L-cysteine
  MOD_RES 21: note = s-(tert-butylthio)-L-cysteine
XPKKKKKVGHHHHCHHCCHHCGSPHHD

SEQ ID NO: 27 - protein, 20 aa, synthetic construct
  LIPID 20: note = 12-aminododecanoic acid
  SITE 1: note = fluorenyl methoxycarbonyl-Proline
  MOD_RES 13: note = s-(tert-butylthio)-L-cysteine
  MOD_RES 16..17: note = s-(tert-butylthio)-L-cysteine
  MOD_RES 20: note = s-(tert-butylthio)-L-cysteine
PKKKRKVGHHHHCHHCCHHC

SEQ ID NO: 28 - protein, 27 aa, synthetic construct
  LIPID 1: note = 12-aminododecanoic acid
  MOD_RES 14: note = s-(tert-butylthio)-L-cysteine
  MOD_RES 17..18: note = s-(tert-butylthio)-L-cysteine
  MOD_RES 21: note = s-(tert-butylthio)-L-cysteine
XPKKKRKVGHHHHCHHCCHHCGSPHHD

SEQ ID NO: 29 - protein, 8 aa, synthetic construct
  LIPID 8: note = 12-aminododecanoic acid
  MOD_RES 4..5: note = s-(tert-butylthio)-L-cysteine
  MOD_RES 8: note = s-(tert-butylthio)-L-cysteine
CHHCCHHC

SEQ ID NO: 30 - protein, 12 aa, synthetic construct
  LIPID 12: note = 12-aminododecanoic acid
  SITE 1: note = fluorenyl methoxycarbonyl-Proline
PKKKRKVGHHHH

SEQ ID NO: 31 - protein, 12 aa, synthetic construct
  SITE 1: note = fluorenyl methoxycarbonyl-Proline
  LIPID 12: note = 12-aminododecanoic acid
PKKKKKVGHHHH

SEQ ID NO: 32 - RNA, 720 nt, synthetic construct
  misc_feature 1..720: note = mRNA
  source 1..720: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 33 - protein, 12 aa, synthetic construct
  MOD_RES 4..5: note = S-Benzyl-L-cysteine
  LIPID 1: note = 12-aminododecanoic acid
LLHCCHLLGSPD

SEQ ID NO: 34 - protein, 14 aa, synthetic construct
  MOD_RES 4..5: note = S-Benzyl-L-cysteine
  LIPID 1: note = 12-aminododecanoic acid
LLHCCHLLGSPHHD

SEQ ID NO: 35 - protein, 14 aa, synthetic construct
  MOD_RES 4..5: note = S-Benzyl-L-cysteine
  LIPID 1: note = 12-aminododecanoic acid
LLHCCHLLGSPHHD

SEQ ID NO: 36 - protein, 18 aa, synthetic construct
  LIPID 1: note = 12-aminododecanoic acid
  MOD_RES 5: note = s-(tert-butylthio)-L-cysteine
  MOD_RES 8..9: note = s-(tert-butylthio)-L-cysteine
  MOD_RES 12: note = s-(tert-butylthio)-L-cysteine
HHHHCHHCCHHCGSPHHD

SEQ ID NO: 37 - protein, 6 aa, synthetic construct
  LIPID 1: note = 12-aminododecanoic acid
GSPHHD

SEQ ID NO: 38 - protein, 18 aa, synthetic construct
  LIPID 1: note = 12-aminododecanoic acid
PKKKRKVGHHHHGSPHHD

SEQ ID NO: 39 - protein, 14 aa, synthetic construct
  LIPID 1: note = 12-aminododecanoic acid
  MOD_RES 1: note = s-(tert-butylthio)-L-cysteine
  MOD_RES 4..5: note = s-(tert-butylthio)-L-cysteine
  MOD_RES 8: note = s-(tert-butylthio)-L-cysteine
CHHCCHHCGSPHHD

SEQ ID NO: 40 - protein, 7 aa, synthetic construct
PKKKRKV